SEQUENCE LISTING (4 sequences)

SEQ ID NO: 1 - DNA, 1434 bp, Mus musculus
  source 1..1434: mol_type = genomic DNA
atggcagagatggggagcaagggggtgacggcggggaagatcgccagcaacgtacagaagaagctgacccgagcgcaggagaaggtcctgcagaaactggggaaggcggacgagacgaaggacgagcagtttgagcagtgtgtccagaacttcaataagcagctgacagagggtacccggctgcagaaggatcttcggacctatctggcttctgttaaagcgatgcacgaagcctccaagaagctgagtgagtgtcttcaggaggtgtatgagcccgagtggcctggcagggatgaagcaaacaagattgcagagaacaatgacctactctggatggactaccaccagaagctggtggaccaggctctgctgaccatggacacctacctaggccagttccctgatatcaagtcgcgcattgccaagcgggggcggaagctggtggactatgacagtgcccggcaccactatgagtctcttcaaaccgccaaaaagaaggatgaagccaaaattgccaaggcagaagaggagctcatcaaagcccagaaggtgttcgaggagatgaacgtggatctgcaggaggagctgccatccctgtggaacagccgtgtaggtttctatgtcaacacgttccagagcatcgcgggtctggaggaaaacttccataaagagatgagtaagctcaatcagaacctcaatgatgtcctggtcagcctagagaagcagcacgggagcaacaccttcacagtcaaggcccaacccagtgacaatgcccctgagaaagggaacaagagcccgtcacctcctccagatggctcccctgctgctacccctgagatcagagtgaaccatgagccagagccggccagtggggcctcacccggggctaccatccccaagtccccatctcagctccggaaaggcccacctgtccctccgcctcccaaacacaccccatccaaggagatgaagcaggagcagattctcagcctttttgatgacgcatttgtccctgagatcagcgtgaccaccccctcccagccagcagaggcctccgaggtggtgggtggagcccaggagccaggggagacagcagccagtgaagcaacctccagctctcttccggctgtggtggtggagaccttctccgcaactgtgaatggggcggtggagggcagcgctgggactggacgcttggacctgcccccgggattcatgttcaaggttcaagcccagcatgattacacggccactgacactgatgagctgcaactcaaagctggcgatgtggtgttggtgattcctttccagaacccagaggagcaggatgaaggctggctcatgggtgtgaaggagagcgactggaatcagcacaaggaactggagaaatgccgcggcgtcttcccggagaattttacagagcgggtgcagtga

SEQ ID NO: 2 - DNA, 1449 bp, Homo sapiens
  source 1..1449: mol_type = genomic DNA
atggcagagatgggcagtaaaggggtgacggcgggaaagatcgccagcaacgtgcagaagaagctcacccgcgcgcaggagaaggttctccagaagctggggaaggcagatgagaccaaggatgagcagtttgagcagtgcgtccagaatttcaacaagcagctgacggagggcacccggctgcagaaggatctccggacctacctggcctccgtcaaagccatgcacgaggcttccaagaagctgaatgagtgtctgcaggaggtgtatgagcccgattggcccggcagggatgaggcaaacaagatcgcagagaacaacgacctgctgtggatggattaccaccagaagctggtggaccaggcgctgctgaccatggacacgtacctgggccagttccccgacatcaagtcacgcattgccaagcgggggcgcaagctggtggactacgacagtgcccggcaccactacgagtcccttcaaactgccaaaaagaaggatgaagccaaaattgccaaggccgaggaggagctcatcaaagcccagaaggtgtttgaggagatgaatgtggatctgcaggaggagctgccgtccctgtggaacagccgcgtaggtttctacgtcaacacgttccagagcatcgcgggcctggaggaaaacttccacaaggagatgagcaagctcaaccagaacctcaatgatgtgctggtcggcctggagaagcaacacgggagcaacaccttcacggtcaaggcccagcccagtgacaacgcgcctgcaaaagggaacaagagcccttcgcctccagatggctcccctgccgccacccccgagatcagagtcaaccacgagccagagccggccggcggggccacgcccggggccaccctccccaagtccccatctcagctccggaaaggcccaccagtccctccgcctcccaaacacaccccgtccaaggaagtcaagcaggagcagatcctcagcctgtttgaggacacgtttgtccctgagatcagcgtgaccaccccctcccagccagcagaggcctcggaggtggcgggtgggacccaacctgcggctggagcccaggagccaggggagacggcggcaagtgaagcagcctccagctctcttcctgctgtcgtggtggagaccttcccagcaactgtgaatggcaccgtggagggcggcagtggggccgggcgcttggacctgcccccaggtttcatgttcaaggtacaggcccagcacgactacacggccactgacacagacgagctgcagctcaaggctggtgatgtggtgctggtgatccccttccagaaccctgaagagcaggatgaaggctggctcatgggcgtgaaggagagcgactggaaccagcacaaggagctggagaagtgccgtggcgtcttccccgagaacttcactgagagggtcccatga

SEQ ID NO: 3 - DNA, 111 bp, synthetic construct
  source 1..111: mol_type = other DNA
tacccagctttcttgtacaaagtggttgatctagagggcccgcggttcgaaggtaagcctatccctaaccctctcctcggtctcgattctacgcgtaccggttagtaatga

SEQ ID NO: 4 - DNA, 1542 bp, synthetic construct
  source 1..1542: mol_type = other DNA
atggcagagatggggagcaagggggtgacggcggggaagatcgccagcaacgtacagaagaagctgacccgagcgcaggagaaggtcctgcagaaactggggaaggcggacgagacgaaggacgagcagtttgagcagtgtgtccagaacttcaataagcagctgacagagggtacccggctgcagaaggatcttcggacctatctggcttctgttaaagcgatgcacgaagcctccaagaagctgagtgagtgtcttcaggaggtgtatgagcccgagtggcctggcagggatgaagcaaacaagattgcagagaacaatgacctactctggatggactaccaccagaagctggtggaccaggctctgctgaccatggacacctacctaggccagttccctgatatcaagtcgcgcattgccaagcgggggcggaagctggtggactatgacagtgcccggcaccactatgagtctcttcaaaccgccaaaaagaaggatgaagccaaaattgccaaggcagaagaggagctcatcaaagcccagaaggtgttcgaggagatgaacgtggatctgcaggaggagctgccatccctgtggaacagccgtgtaggtttctatgtcaacacgttccagagcatcgcgggtctggaggaaaacttccataaagagatgagtaagctcaatcagaacctcaatgatgtcctggtcagcctagagaagcagcacgggagcaacaccttcacagtcaaggcccaacccagtgacaatgcccctgagaaagggaacaagagcccgtcacctcctccagatggctcccctgctgctacccctgagatcagagtgaaccatgagccagagccggccagtggggcctcacccggggctaccatccccaagtccccatctcagctccggaaaggcccacctgtccctccgcctcccaaacacaccccatccaaggagatgaagcaggagcagattctcagcctttttgatgacgcatttgtccctgagatcagcgtgaccaccccctcccagccagcagaggcctccgaggtggtgggtggagcccaggagccaggggagacagcagccagtgaagcaacctccagctctcttccggctgtggtggtggagaccttctccgcaactgtgaatggggcggtggagggcagcgctgggactggacgcttggacctgcccccgggattcatgttcaaggttcaagcccagcatgattacacggccactgacactgatgagctgcaactcaaagctggcgatgtggtgttggtgattcctttccagaacccagaggagcaggatgaaggctggctcatgggtgtgaaggagagcgactggaatcagcacaaggaactggagaaatgccgcggcgtcttcccggagaattttacagagcgggtgcagtacccagctttcttgtacaaagtggttgatctagagggcccgcggttcgaaggtaagcctatccctaaccctctcctcggtctcgattctacgcgtaccggttagtaatga

(19) United States
(12) Patent Application Publication
(10) Pub. No.: US 2025/0381306 A1
(43) Pub. Date: Dec. 18, 2025

(54) COMPOSITIONS AND METHODS FOR TREATING DIABETIC CARDIOMYOPATHY

(71) Applicant: UNIVERSITY OF UTAH RESEARCH FOUNDATION, Salt Lake City, UT (US)

(72) Inventors: TingTing HONG, Salt Lake City, UT (US); Robin M. SHAW, Salt Lake City, UT (US); Jing LI, Salt Lake City, UT (US)

(21) Appl. No.: 19/142,456

(22) PCT Filed: Dec. 13, 2023
(86) PCT No.: PCT/US2023/083815

§ 371 Date: Jun. 23, 2025

Related U.S. Application Data
(60) Provisional application No. 63/476,735, filed on Dec. 22, 2022.
(60) Provisional application No. 63/440,853, filed on Jan. 24, 2023.

(51) Int. Cl. A61K 48/00; C12N 15/86

(52) U.S. Cl. CPC A61K 48/0058; C12N 15/86; C12N 2750/14143

(57) ABSTRACT

Described herein are compositions and methods for treating, preventing, reducing the likelihood of having, reducing the severity of and/or slowing the progression of a medical condition related to diabetic cardiomyopathy in a subject using cardiac bridging integrator 1 (cBIN1) as a therapeutic agent. In one embodiment, the compositions and methods comprise a cBIN1 gene therapy. In another embodiment, the disclosed compositions and methods may reduce blood glucose levels in a subject.

### CROSS-REFERENCE TO RELATED APPLICATIONS

This application claims priority to U.S. Provisional Patent Application Nos. 63/440,853, filed on Jan. 24, 2023, and 63/476,735, filed on Dec. 22, 2022, each of which is incorporated by reference herein in its entirety.

### FEDERALLY SPONSORED RESEARCH

This invention was made with government support under grants HL133286, HL159983, and AG074593 awarded by the National Institutes of Health. The government has certain rights in this invention.

### REFERENCE TO SEQUENCE LISTING

This application was filed with a Sequence Listing XML in ST.26 XML format in accordance with 37 C.F.R. § 1.821. The Sequence Listing XML file submitted in the USPTO Patent Center, “026389-0003-WO01_sequence_listing_XML_19 Nov. 2023.xml,” was created on Nov. 19, 2023, contains 4 sequences, has a file size of 8.9 Kbytes, and is hereby incorporated by reference in its entirety into the specification.

### TECHNICAL FIELD

Described herein are compositions and methods for treating, preventing, reducing the likelihood of having, reducing the severity of and/or slowing the progression of a medical condition related to diabetic cardiomyopathy in a subject using cardiac bridging integrator 1 (cBIN1) as a therapeutic agent. In one embodiment, the compositions and methods comprise a cBIN1 gene therapy. In another embodiment, the disclosed compositions and methods may reduce blood glucose levels in a subject.

### BACKGROUND

Diabetic cardiomyopathy (DCM) is an increasing global epidemic, affecting over one third of patients with diabetes. DCM is a major cause of heart failure and cardiac premature mortality and is associated with hyperglycemia, insulin resistance, intra-cardiomyocyte calcium mishandling, and mitochondrial dysfunction. Existing therapeutic options for treating DCM are limited and inadequate, and effective therapies to prevent and rescue DCM are a major global unmet need. Furthermore, most patients with diabetic cardiomyopathy have symptoms that manifest as heart failure with preserved ejection fraction (HFpEF), and the diagnostic tools and therapeutic options for this population are even more limited.

Known pathogenic and potentially targetable mechanisms of DCM in myocardium include impaired insulin metabolic signaling, intracellular calcium dysregulation, mitochondrial dysfunction, and oxidative stress. In non-diabetic failing cardiomyocytes, the calcium handling microdomains at transverse-tubules (t-tubules) that are organized by the membrane scaffolding protein, cardiac bridging integrator 1 (cBIN1), are an emerging therapeutic target. In the cardiac system, the alternate splicing of the Bin1 gene produces 4-6 transcript variants and the corresponding protein isoforms. The two cardiac characteristic isoforms feature the inclusion of exon 13 (deficient of exons 14-16) with or without the ubiquitously alternatively spliced exon 17 to form the protein isoforms of BIN1+exon 13 and BIN1+exon 13+exon 17 (called cBIN1). Of these two cardiac isoforms, cBIN1 containing exons 13 and 17 is the isoform which localizes to cardiac t-tubules and acts as a critical membrane scaffolding protein. However, the remodeling and therapeutic targeting of cBIN1 microdomains in DCM cardiomyocytes remains unexplored. Further, t-tubule membrane domains are also enriched with glucose transporter 4 (GLUT4) to allow for insulin-induced glucose uptake, providing carbohydrate fuel to adjacent mitochondria.

What is needed are new compositions and methods for treating DCM in patients with acquired or genetic predisposition. Such compositions and methods may be useful in treating a variety of DCM-associated syndromes, including heart failure and diabetes.

### SUMMARY

One embodiment described herein is a method of treating, preventing, reducing the likelihood of having, reducing the severity of, and/or slowing the progression of diabetic cardiomyopathy in a subject, the method comprising: administering to the subject a therapeutically effective amount of a pharmaceutical composition comprising: a cardiac bridging integrator 1 (cBIN1) gene expression vector comprising a cBIN1 polynucleotide sequence encoding a cBIN1 polypeptide or functional variant or fragment thereof. In one aspect, the pharmaceutical composition is administered to the subject by intravenous (i.v.) injection. In another aspect, the pharmaceutical composition is administered to the subject by cardiac catheter infusion to myocardium. In another aspect, the cBIN1 polynucleotide sequence comprises DNA, RNA, or a combination thereof. In another aspect, the cBIN1 polynucleotide sequence has at least 90-99% identity to any one of SEQ ID NO: 1, 2, or 4. In another aspect, the cBIN1 polynucleotide sequence is any one of SEQ ID NO: 1, 2, or 4. In another aspect, the cBIN1 gene expression vector is selected from a non-viral vector, a viral vector, an adeno-associated virus (AAV) vector, a recombinant AAV (rAAV) vector, a single-stranded AAV vector, a double-stranded AAV vector, a self-complementary AAV (scAAV) vector, or combinations thereof. In another aspect, the cBIN1 gene expression vector is an AAV vector of a serotype selected from AAV1, AAV2, AAV3, AAV4, AAV5, AAV6, AAV7, AAV8, AAV9, AAV10, AAV11, AAV12, AAVrh74, a hybrid serotype thereof, or a derivative thereof. In another aspect, the cBIN1 gene expression vector is an AAV9 vector. In another aspect, the cBIN1 gene expression vector is a muscle-tropic AAV vector. In another aspect, the non-viral vector comprises a lipid carrier, an exosome, a polymer-based carrier, a chemical-based carrier, a conjugated carrier, or combinations thereof. In another aspect, the pharmaceutical composition is administered to the subject using a dosing regimen based on cBIN1 gene expression vector genome (vg) per kg body weight of the subject. In another aspect, the pharmaceutical composition is administered to the subject at a dose ranging from about 5×10¹¹ vg/kg to about 5×10¹³ vg/kg cBIN1 gene expression vector. In another aspect, the subject has type 1 diabetes or type 2 diabetes. In another aspect, the subject has diabetic cardiomyopathy. In another aspect, the pharmaceutical composition reduces blood glucose levels in the subject. In another aspect, the pharmaceutical composition normalizes or restores the intracellular distribution of calcium handling machinery in myocardium of the subject, wherein the calcium handling machinery comprises one or more of SERCA2a, Cav1.2, or RyR₂. In another aspect, the pharmaceutical composition rehabilitates or increases transverse-tubule microfolds or microdomains in myocardium of the subject. In another aspect, the pharmaceutical composition selectively expresses the cBIN1 polypeptide or functional variant or fragment thereof in the heart of the subject. In another aspect, the pharmaceutical composition increases selective expression of the cBIN1 polypeptide or functional variant or fragment thereof in the heart of the subject by at least about 20% relative to an endogenous cBIN1 expression level in the heart of the subject. In another aspect, the pharmaceutical composition expresses the cBIN1 polypeptide or functional variant or fragment thereof in the subject for at least 6 months. In another aspect, the subject is a mammal. In another aspect, the subject is a human.

Another embodiment described herein is a pharmaceutical composition comprising a cardiac bridging integrator 1 (cBIN1) gene expression vector comprising a cBIN1 polynucleotide sequence encoding a cBIN1 polypeptide or functional variant or fragment thereof. In one aspect, the cBIN1 polynucleotide sequence comprises DNA, RNA, or a combination thereof. In another aspect, the cBIN1 polynucleotide sequence has at least 90-99% identity to any one of SEQ ID NO: 1, 2, or 4. In another aspect, the cBIN1 polynucleotide sequence is any one of SEQ ID NO: 1, 2, or 4. In another aspect, the cBIN1 gene expression vector is selected from a non-viral vector, a viral vector, an adeno-associated virus (AAV) vector, a recombinant AAV (rAAV) vector, a single-stranded AAV vector, a double-stranded AAV vector, a self-complementary AAV (scAAV) vector, or combinations thereof. In another aspect, the cBIN1 gene expression vector is an AAV vector of a serotype selected from AAV1, AAV2, AAV3, AAV4, AAV5, AAV6, AAV7, AAV8, AAV9, AAV10, AAV11, AAV12, AAVrh74, a hybrid serotype thereof, or a derivative thereof. In another aspect, the cBIN1 gene expression vector is an AAV9 vector. In another aspect, the cBIN1 gene expression vector is a muscle-tropic AAV vector. In another aspect, the non-viral vector comprises a lipid carrier, an exosome, a polymer-based carrier, a chemical-based carrier, a conjugated carrier, or combinations thereof.

Another embodiment described herein is the use of a cardiac bridging integrator 1 (cBIN1) gene therapy in a medicament for treating, preventing, reducing the likelihood of having, reducing the severity of, and/or slowing the progression of diabetic cardiomyopathy in a subject. In one aspect, the cBIN1 gene therapy comprises a therapeutically effective amount of a pharmaceutical composition comprising: a cBIN1 gene expression vector comprising a cBIN1 polynucleotide sequence encoding a cBIN1 polypeptide or functional variant or fragment thereof.

Another embodiment described herein is the use of a plasma cardiac bridging integrator 1 (cBIN1) score (CS) to identify a subject having diabetic cardiomyopathy for cBIN1 gene therapy treatment. In one aspect, the cBIN1 gene therapy treatment comprises a therapeutically effective amount of a pharmaceutical composition comprising: a cBIN1 gene expression vector comprising a cBIN1 polynucleotide sequence encoding a cBIN1 polypeptide or functional variant or fragment thereof. In another aspect, the plasma CS is a non-invasive measure of target engagement and therapeutic response of the subject to the cBIN1 gene therapy treatment. In another aspect, the plasma CS is the natural log of the ratio of a median plasma cBIN1 concentration in a normal human population to a measured cBIN1 concentration in the subject having diabetic cardiomyopathy.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-I show that cBIN1 is downregulated in diabetic mouse hearts and can be rescued by exogenous AAV9-cBIN1 to improve exercise capacity and systemic blood glucose control. FIG. 1A shows a schematic illustration of the experimental protocol used. FIG. 1B shows bar graphs of plasma cBIN1 score (CS) values in 9-week-old pre-treated (left) and 17-week-old post-treated (right) db/db mice and littermate control db/m mice (n=11-16). FIG. 1C shows the maximal distance run (m) during mouse exercise tolerance tests in each group before (left, n=24-28) and after (right, n=16-17) treatment. FIG. 1D-E show plasma glucose (FIG. 1D) and insulin (FIG. 1E) levels (non-fasting) in pre-treated mice (left, n=9-14) and post-treated mice (right, n=15-17). FIG. 1F shows the percent of peak blood glucose during intraperitoneal glucose tolerance tests (iGTT) in AAV9-GFP/cBIN1 treated mice at 17 weeks of age with intraperitoneal injection of glucose (1 g/kg) after 16-hour fasting (n=4-6 mice per group). FIG. 1G shows the percentage of baseline blood glucose during intraperitoneal insulin tolerance tests (iITT) in AAV9-GFP/cBIN1 treated mice at 17 weeks of age with intraperitoneal injection of insulin (0.75 IU/kg) after 4-hour fasting (n=4-6 animals per group). FIG. 1H shows quantitative real-time PCR (qPCR) analysis of V5-tagged exogenous cBin1 (V5 normalized to the housekeeping gene Hprt1 and then compared with non-injected control organs) in indicated organs from db/db mice injected with AAV9-cBIN1-V5. SKM, skeletal muscle; Pan, pancreas; K, kidney; Sp, spleen; and Adi, adipose tissue (n=4 animals/group). FIG. 1I shows western blot analysis of cBIN1 and GAPDH with quantification (cBIN1/GAPDH) in heart lysates from post-treatment mice (n=15 hearts per group). Data are presented as mean±SEM. Unpaired 2-tailed Student's t test or nonparametric Mann-Whitney U test was used for two group comparison between db/m and db/db. *, *** indicates p<0.05, p<0.001, respectively, for comparison vs. db/m (FIG. 1B-E) or no virus controls (FIG. 1H). One-way ANOVA followed by Bonferroni's test or nonparametric Kruskal-Wallis test followed by Dunn's test was used for comparison between selected pairs (FIGS. 1B-E and 11). Two-way ANOVA followed by Tukey's test was used for comparison on multiple timepoints among treatment groups (FIG. 1F-G). *, **, *** indicates p<0.05, p<0.01, and p<0.001, respectively, for comparison vs. db/m+GFP; † indicates p<0.05 for comparison between db/db+GFP and db/db+cBIN1.

FIG. 2A-E show that exogenous cBIN1 normalizes membrane microdomains at t-tubules. FIG. 2A shows representative transmission electron microscopy images of post-treatment hearts (scale bar=1 μm) from each group (top), and quantification of the degree of contour of t-tubules (bottom; n=100-101 t-tubules from 16-40 images of 2-3 myocardial sections and 3 hearts from each group). Data are presented as the percentage of t-tubules. Chi-square test was used to compare t-tubule contour between groups. *** indicates p<0.001 for db/m+GFP vs. db/db+GFP; ^{†††} indicates p<0.001 for db/db+GFP vs. db/db+cBIN1. FIG. 2B shows western blot analysis of cBIN1, SERCA2a, Cav1.2, RyR₂, GLUT4, and IRAP in total cardiac microsome and sucrose-gradient isolated TT/jSR fraction (F4) from each group (n=5 hearts per group). FIG. 2C shows representative spinning disc confocal images of post-treatment mouse myocardium with power spectrum analysis of boxed areas, and quantification of SERCA2a peak power density at t-tubules (n=50-74 cells from 3 hearts per group). FIG. 2D shows representative GLUT4 and IRAP confocal images in post-treatment isolated cardiomyocytes with corresponding fluorescence intensity profiles, and quantification of GLUT4 and IRAP peak power density at t-tubules (n=50-74 cells from 3 hearts) (scale bar=10 μm). Data are presented as mean±SEM. One-way ANOVA or Kruskal-Wallis test followed by Fisher LSD test was used for multiple comparison among groups. *, **, *** indicates p<0.05, p<0.01, and p<0.001 for comparison vs. db/m+GFP; ^{†}, ^{††}, ^{†††} indicates p<0.05, p<0.01, and p<0.001 for comparison between db/db+GFP and db/db+cBIN1. FIG. 2E shows glucose uptake following insulin (0 and 10 nM) stimulation in cardiomyocytes (CMCs) isolated from each group (n=10-12 repeats from 4 animals per group). 20 μM metformin (Met) was included as a positive control treatment. Two-way ANOVA followed by Fisher LSD test for multiple comparisons was used for multiple comparison among groups. *** indicates p<0.001 for comparison vs. db/m+GFP with 10 nM insulin treatment; ^{††}, ^{†††} indicates p<0.01 and p<0.001 for comparison vs. db/db+GFP with 10 nM insulin treatment. #, ##, ###indicates p<0.05, p<0.01, and p<0.001 for comparison of 10 nM insulin vs. 0 nM insulin baseline within each therapeutic group.

FIG. 3A-F show that AAV9-cBIN1 downregulates myocardial mitochondrial components and improves mitochondrial function in diabetic cardiomyocytes. FIG. 3A shows bar graphs of LFQ LC-MS/MS data (fold changes over control db/m mice) of SERCA2a, RyR₂, GLUT4, IRAP, RAB5a, and RAB10 from the db/m mice treated with AAV9-GFP (n=4 hearts) and db/db mice treated with AAV9-GFP (n=4 hearts) or cBIN1 (n=3 hearts). FIG. 3B shows a PCA plot of all three groups (n=2 repeats/heart×3-4 hearts/group), generated based on LFQ LC-MS/MS proteomics. FIG. 3C shows STRING enrichment analysis of the top 10 most significantly regulated proteins for gene ontology (GO) terms for cellular component (left), KEGG pathways (center), and reactome pathways (right). FIG. 3D-E show representative JH₂O₂/JO₂ (FIG. 3D) and calcium retention curve with quantified calcium retention capacity (FIG. 3E) produced from isolated cardiac mitochondria obtained from each group after treatment (n=5-6 hearts per group). FIG. 3F shows seahorse analysis of oxygen consumption rate in isolated cardiomyocytes from each group after treatment. Bar graphs on the right include quantification of maximal and spare oxygen consumption rate (OCR) from each group (n=15 repeats per group). All data are presented as mean±SEM. *, **, *** indicates p<0.05, p<0.01, and p<0.001 for comparison vs. db/m+GFP; ^{†}, ^{††}, ^{†††} indicates p<0.05, p<0.01, and p<0.001 for comparison between db/db+GFP and db/db+cBIN1.

FIG. 4A-E show that exogenous cBIN1 rescues diastolic heart failure in diabetic mice. FIG. 4A shows representative images of longitudinal axis view of left ventricles at end diastolic (top) and systolic (bottom) phases in post-treatment mice (17 weeks of age). Quantification of end-diastolic volume (EDV), cardiac output (CO), and ejection fraction (EF) from each group are included in the bar graphs (n=12-17). FIG. 4B shows representative images of mitral valve inflow pulsed wave Doppler (top) and septal mitral valve annulus tissue Doppler (bottom) in post-treatment mice. Quantification of E/A, E/e′, and isovolumic relaxation time (IVRT) from each group are included in the bar graphs (n=11-17). FIG. 4C-D show the ratio of heart weight (FIG. 4C) and lung weight (FIG. 4D) over tibial length after treatment (n=13-17). FIG. 4E shows a schematic illustration of calcium handling, GSV translocation, and mitochondrial alteration regulated by cBIN1 microdomains at t-tubules in healthy and diabetic failing cardiomyocytes. All data are presented as mean±SEM. *, **, *** indicates p<0.05, p<0.01, and p<0.001 for comparison vs. db/m+GFP; ^{†}, ^{††}, ^{†††} indicates p<0.05, p<0.01, and p<0.001 for comparison between db/db+GFP and db/db+cBIN1.

FIG. 5A-F show that db/db mice develop diastolic dysfunction at 9 weeks of age. FIG. 5A shows representative mitral valve inflow pulsed wave Doppler images (top) and tissue Doppler images of septal mitral valve annulus (bottom). FIG. 5B shows a longitudinal axis view of left ventricles at end diastolic (top) and end systolic (bottom) phase in 9-week-old db/m and db/db mice. FIG. 5C-F show quantitative analysis of E/A (FIG. 5C), E/e′ (FIG. 5D), isovolumic relaxation time (IVRT) (FIG. 5E), and end-diastolic volume (EDV) (FIG. 5F) (n=24-35). All data are presented as mean±SEM. Unpaired Student's t-test was used for comparison between db/m and db/db. *, *** indicates p<0.05 and p<0.001 for comparison vs. db/m.

FIG. 6A-D show fluorescent images of myocardium from mice injected with V5-tagged-AAV9 confirming effective exogenous protein transduction. Representative immunostaining images of myocardial tissue stained with rabbit anti-V5 antibody (green) and DAPI (blue) are shown for negative control (FIG. 6A), db/m+GFP (FIG. 6B), db/db+GFP (FIG. 6C), and db/db+cBIN1 (FIG. 6D) mice. V5-tagged exogenous protein was transduced into nearly 100% of myocardium by the AAV9 virus. The negative control from mice without AAV9 injection was treated with the same immunofluorescent labeling procedure including primary and secondary antibodies. Scale bar=50 μm.

FIG. 7 shows protein expression of calcium handling and GLUT4 translocation proteins in AAV9-treated mice for microsome and F1-F4 fractions following sucrose gradient fractionation. Representative western blots of total RyR₂, Cav1.2, SERCA2a, GLUT4, IRAP, and cBIN1 protein expression are shown from db/m+GFP, db/db+GFP, and db/db+cBIN1 hearts.

FIG. 8A-B show protein expression of calcium handling and GLUT4 translocation proteins in AAV9-treated mice. Representative western blots of total RyR₂, Cav1.2, and SERCA2a (FIG. 8A) and of total GLUT4 and IRAP (FIG. 8B) protein expression in myocardial tissue are shown from db/m+GFP, db/db+GFP, and db/db+cBIN1 hearts at 17 weeks, with quantification shown to the right in the bar graphs (n=5-10 hearts per group).

FIG. 9A-D show that AAV9-cBIN1 downregulates myocardial mitochondrial components through promoting mitophagy in diabetic cardiomyocytes. FIG. 9A shows a heatmap of the top 30 most significantly changed proteins comparing db/db+GFP and db/db+cBIN1 groups. FIG. 9B shows real-time quantitative PCR analysis of mitochondrial DNA in myocardial tissue from post-treatment mice. FIG. 9C-D show representative western blot images (top) and quantification (bottom) of PINK1 (FIG. 9C) and LC3-I/II (FIG. 9D) in heart lysates from post-treatment mice from each group (n=6-7 heats per group). Data are presented as mean±SEM. *, **, *** indicates p<0.05, p<0.01, and p<0.001 for comparison vs. db/m+GFP; †, †† indicates p<0.05 and p<0.01 for comparison between db/db+GFP and db/db+cBIN1.

FIG. 10A-E show that Bin1 deletion in cardiomyocytes impairs GLUT4 translocation and glucose utilization, weakening systemic glucose response to insulin in mice. FIG. 10A shows representative GLUT4 confocal images in isolated cardiomyocytes from WT and Bin1 HT mice with or without prior AAV9-cBIN1 rescue. Cardiomyocytes were treated with 0 or 10 nM insulin for 20 minutes before collection. The images of the boxed areas with the corresponding fluorescence intensity profiles are included in the bottom panels. Scale bar: 10 μm. FIG. 10B shows quantification of GLUT4 peak power density at t-tubules (n=30-47 cells from 3 hearts per group) (left), and 2-deoxy-D-glucose (2DG) uptake following insulin (0 and 10 nM) stimulation in cardiomyocytes isolated from each group (n=10-12 repeats from 3 animals per group) (right). FIG. 10C shows ¹³C enrichment of extracellular lactate and intracellular glycolysis and TCA cycle intermediates from cardiomyocytes isolated from each group (n=3-5). A schematic for the metabolic pathway of ¹³C₆-glucose is included. Pyr., pyruvate; Lac., lactate; Glc., glucose; F6P, fructose-6-phophate; F1,6BP, fructose-1,6-biphosphate; DHAP, dihydroxyacetone phosphate; 3PG, 3-phosphoglyceric acid; PEP, phosphoenolpyruvate; Cit., citrate; β-KG, alpha-ketoglutarate; Succ., succinate; Fum., fumarate; and Mal., malate. FIG. 10D shows the percent of peak blood glucose during iGTT in each group (n=4). FIG. 10E shows the percent of baseline blood glucose during iITT in each group (n=4). Data are presented as mean±SEM. Two-way ANOVA followed by Tukey's (3 treatment groups, FIG. 10B-E) or Bonferroni's (2 insulin doses, FIG. 10B) test was used. *, **, *** indicates P<0.05, 0.01, 0.001, respectively, for comparison versus WT+PBS (FIG. 10B-E); †, ††, ††† indicates P<0.05, 0.01, 0.001, respectively, for comparison between Bin1 HT+PBS and Bin1 HT+cBIN1 (FIG. 10B-E); and #, ###indicates P<0.05, 0.001, respectively, for comparison of 10 nM versus 0 nM insulin within each group (FIG. 10B).

FIG. 11 shows that AAV9-cTnT-cBIN1 improves systemic glucose tolerance in diabetic mice. The percent of peak blood glucose during iGTT in AAV9-cTnT-GFP/cBIN1-treated mice is shown following an i.p. injection of glucose (1 g/kg) after 12-hour fasting (n=10 mice per group). Data are presented as mean±SEM. Two-way ANOVA followed by Tukey's for multiple comparisons among groups was used. *, ** *** indicates P<0.05, 0.01, 0.001, respectively, for comparison versus db/m+GFP; †† indicates P<0.01 for comparison between db/db+GFP and db/db+cBIN1.

FIG. 12A-B show that intravenous (i.v.) administration of AAV9-cBIN1 (6×10¹¹ vg/kg) transduces exogenous cBIN1 expression in minipig hearts. FIG. 12A shows cardiac expression of exogenous cBin1-V5 (ΔΔCt of V5/HPRT1 when compared with PBS controls) in minipig hearts 6 months after AAV9-cBIN1 i.v. injection. FIG. 12B shows exogenous cBin1-V5 as a percentage of endogenous porcine cBIN1 (derived from ΔCt of V5/cBIN1) in minipig hearts 6 months after AAV9-cBIN1 i.v. injection (n=10 tissue samples across left ventricle obtained from 2 minipigs per group). Data are presented as mean±SEM. Unpaired 2-tailed Student's t test or Mann-Whitney U test was used. *** indicates P<0.001 for comparison versus PBS controls.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid or polynucleotide may be DNA, both genomic and cDNA, RNA (e.g., mRNA), or a hybrid thereof, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, “variants” can include, but are not limited to, those that include conservative amino acid (AA) substitution, SNP variants, degenerate variants, and biologically active portions of a gene. A “degenerate variant” as used herein refers to a variant that has a mutated nucleotide sequence, but still encodes the same polypeptide due to the redundancy of the genetic code. There are 20 naturally occurring amino acids; however, some of these share similar characteristics. For example, leucine and isoleucine are both aliphatic, branched, and hydrophobic. Similarly, aspartic acid and glutamic acid are both small and negatively charged. Conservative substitutions in proteins often have a smaller effect on function than non-conservative mutations. Although there are many ways to classify amino acids, they are often sorted into six main groups on the basis of their structure and the general chemical characteristics of their R groups. A mutation among the same class of amino acids is considered a conservative amino acid substitution.

The term “functional” when used in conjunction with “variant” or “fragment” refers to an entity or molecule which possess a biological activity that is substantially similar to a biological activity of the entity or molecule of which it is a variant or fragment thereof. In accordance with the present invention, a cBIN1 polynucleotide sequence encoding a cBIN1 polypeptide or functional variant or fragment thereof may be modified, for example, to facilitate or improve identification, expression, isolation, storage and/or administration, so long as such modifications do not reduce its function to an unacceptable level. In various embodiments, a cBIN1 polypeptide functional variant or fragment thereof has at least 10%, 20%, 30%, 40%, 50%, 60%, 70%, 80%, 90%, 95%, or 99% of the function of a full-length wildtype cBIN1 polypeptide.

As used herein, “substantial identity” of polynucleotide sequences means that a polynucleotide comprises a sequence that has at least 25% sequence identity compared to a reference sequence as determined using programs known in the art (e.g., Basic Local Alignment Search Tool (BLAST)). In preferred embodiments, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present invention encoding a protein or polypeptide of the present invention include nucleic acid sequences that have substantial identity to the nucleic acid sequences that encode the proteins or polypeptides of the present invention. Polynucleotides encoding a polypeptide comprising an amino acid sequence that has at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

As used herein, “substantial identity” of amino acid sequences (and of polypeptides having these amino acid sequences) means that an amino acid sequence comprises a sequence that has at least 25% sequence identity compared to a reference sequence as determined using programs known in the art (e.g., BLAST). In preferred embodiments, percent identity can be any integer from 25% to 100%. More preferred embodiments include amino acid or polypeptide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences except that residue positions which are not identical may differ by one or more conservative amino acid changes, as described above. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups include valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present invention, include amino acid sequences that have substantial identity to the amino acid sequences of the reference polypeptide sequences.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, therapeutic, often beneficial, effect. In some embodiments, disclosed compositions may further comprise one or more pharmaceutically acceptable carriers or excipients. Example pharmaceutically acceptable carriers may include, but are not limited to, liposomes, polymeric micelles, microspheres, microparticles, dendrimers, and/or nanoparticles. Example pharmaceutically acceptable excipients may include, but are not limited to, buffering agents, salts, detergents, surfactants, acidifying agents, alkalizing agents, antimicrobial preservatives, antioxidants, chelating agents, and/or solubilizing agents.

As used herein, the terms “control” or “reference” are used herein interchangeably. A “reference” or “control” level may be a predetermined value or range, which is employed as a baseline or benchmark against which to assess a measured result. “Control” also refers to control experiments, conditions, cells, or animals.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the term “administering” refers to the placement of an agent or a composition as disclosed herein into a subject by a method or route which results in at least partial localization of the agents or composition at a desired site. “Route of administration” may refer to any administration pathway known in the art, including but not limited to oral, intravenous (i.v.), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, infusion (e.g., cardiac catheter infusion), intracapsular, intracardiac, intradermal, intramuscular, intraperitoneal, intrapulmonary, intraspinal, intrasternal, intrathecal, intrauterine, intravascular, intravenous (i.v.), intraarterial, subarachnoid, subcapsular, subcutaneous, transmucosal, or transtracheal. Via the parenteral route, the agent or composition may be in the form of solutions or suspensions for i.v. infusion or i.v. injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. In one embodiment, the agent or composition may be provided in a powder form and mixed with a liquid, such as water, to form a beverage. In accordance with the present invention, “administering” can be self-administering. For example, it is considered “administering” when a subject consumes a composition as disclosed herein.

As used herein, “contacting” refers to contacting a target cell with a therapeutic agent (e.g., a cBIN1 gene expression vector or pharmaceutical composition) using any method that is suitable for placing the agent on, in, or adjacent to a target cell. For example, when the cells are in vitro, contacting the cells with the agent can comprise adding the agent to culture medium containing the cells. For example, when the cells are in vivo, contacting the cells with the agent can comprise administering the agent to a subject.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, weight, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments. In some embodiments of the present invention, a subject is in need of treatment if the subject is suffering from, or at risk of suffering from, diabetic cardiomyopathy.

As used herein, “diabetic cardiomyopathy” or “DCM” may arise in a subject having either type 1 diabetes or type 2 diabetes. In some embodiments, patients may develop DCM, or may be at risk of developing DCM, due to acquired and/or genetic predispositions.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic manner. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest. In one embodiment of the present invention, a method of treating, preventing, reducing the likelihood of having, reducing the severity of, and/or slowing the progression of a diabetic cardiomyopathy syndrome in a subject is described. In some embodiments, the disclosed treatment methods may reduce blood glucose levels in a subject.

As used herein, “sample” or “target sample” refers to any sample in which the presence and/or level of a target analyte or target biomarker is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological or bodily fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, “target analyte” or “target biomarker” refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, can be associated with and/or be indicative of a particular state or process. Biomarkers can include, but are not limited to, cells or cellular components (e.g., a viral cell, a bacterial cell, a fungal cell, a cancer cell, etc.), small molecules, lipids, carbohydrates, nucleic acids, peptides, proteins, enzymes, antigens, and antibodies. A biomarker can be derived from an infectious agent, such as a bacterium, fungus or virus, or can be an endogenous molecule that is found in greater or lesser abundance in a subject suffering from a disease or disorder as compared to a healthy individual (e.g., an increase or decrease in expression of a gene or gene product).

The present disclosure describes compositions and methods comprising a cBIN1 gene therapy for the treatment of DCM. In some embodiments, the disclosed cBIN1 gene therapy is capable of rescuing DCM and reducing blood glucose levels in a subject.

Various embodiments of the present invention provide a pharmaceutical composition comprising a cBIN1 gene therapy for treating DCM in a subject. In various embodiments, the cBIN1 gene therapy is of a mammal. In various embodiments, the cBIN1 gene therapy is of a primate, for example, a human, a chimpanzee, a gorilla, or a monkey. In various embodiments, the cBIN1 gene therapy is of a horse, a goat, a donkey, a cow, a bull, or a pig. In various embodiments, the cBIN1 gene therapy is of a rodent, for example, a mouse, a rat, or a guinea pig. In various embodiments, the cBIN1 gene therapy is of a chicken, a duck, a frog, a dog, a cat, or a rabbit.

### Compositions

The present disclosure provides cBIN1 gene therapy pharmaceutical compositions comprising a cBIN1 gene expression vector comprising a cBIN1 polynucleotide sequence encoding a cBIN1 polypeptide or functional variant or fragment thereof. In some embodiments, the disclosed compositions may further comprise one or more pharmaceutically acceptable carriers or excipients.

cBIN1 Polynucleotide Sequences and Gene Expression Vectors

The disclosed pharmaceutical compositions may comprise a cBIN1 gene expression vector comprising a cBIN1 polynucleotide sequence encoding a cBIN1 polypeptide or functional variant or fragment thereof. In some embodiments, the cBIN1 polynucleotide sequence may comprise DNA, RNA (e.g., mRNA), or a combination thereof encoding a cBIN1 polypeptide or functional variant or fragment thereof.

In some embodiments, various gene expression vectors as described herein are used to produce various cBIN1 polypeptides or functional variants or fragments thereof. For example, various gene expression vectors may be introduced into bacteria or yeast to produce various cBIN1 polypeptides or functional variants thereof, which are later isolated. In various embodiments, the gene expression vector is a plasmid.

In various embodiments, the gene expression vector is a non-viral vector, a viral vector, an adeno-associated virus (AAV) vector, a recombinant AAV (rAAV) vector, a single-stranded AAV vector, a double-stranded AAV vector, a self-complementary AAV (scAAV) vector, or a combination thereof. In various embodiments, non-viral vector delivery of the disclosed cBIN1 polynucleotide sequences may comprise the use of non-viral vector carriers including, but not limited to, lipid carriers (e.g., lipid nanoparticles), exosomes, polymer-based carriers, chemical-based carriers, conjugated carriers (e.g., transferrin-conjugated approaches), and the like. In some embodiments, the gene expression vector is a non-viral vector comprising a lipid carrier such as a lipid nanoparticle (LNP). Non-viral vector delivery approaches of the disclosed cBIN1 polynucleotide sequences may also comprise administration of naked polynucleotide sequences (i.e., not protected and/or devoid of a carrier).

In various embodiments, the gene expression vector is a polynucleotide or a virus particle. In various embodiments, the gene expression vector is a virus particle having a serotype of AAV1, AAV2, AAV3, AAV4, AAV5, AAV6, AAV7, AAV8, AAV9, AAV10, AAV11, AAV12, AAVrh74, a hybrid serotype thereof, or a derivative of one of these capsids. In various embodiments, the gene expression vector is a muscle-tropic AAV-based capsid that enables potent and specific muscle-directed targeted delivery of cBIN1 polynucleotide sequences. These muscle-tropic AAV-based capsids may be either derivatives of existing capsids (e.g., AAV9, AAVrh74), or may be newly discovered or custom designed capsids.

In various embodiments, the cBIN1 gene expression vector may be a vector containing a ubiquitous and/or constitutive promoter (e.g., CMV promoter) for ubiquitous and/or constitutive expression of cBIN1. In various embodiments, the cBIN1 gene expression vector may be a vector containing a tissue-specific promoter (e.g., cardiac-specific promoter) for targeted and tissue-specific expression of cBIN1, such as specific expression of cBIN1 in the heart.

In one nonlimiting exemplary embodiment, the cBIN1 gene expression vector is an AAV9 vector containing a CMV promoter for ubiquitous expression, wherein a cBIN1 polynucleotide sequence encoding a cBIN1 polypeptide or functional variant or fragment thereof is cloned into the AAV9 vector.

In another nonlimiting exemplary embodiment, the cBIN1 polynucleotide sequence encoding a cBIN1 polypeptide or functional variant or fragment thereof is the mouse cBIN1 (BIN1+exon 13+exon 17) coding sequence (SEQ ID NO: 1):

Mouse cBIN1 Polynucleotide Sequence
SEQ ID NO: 1
ATGGCAGAGATGGGGAGCAAGGGGGTGACGGCGGGGAAGATCGCCAGCAA
CGTACAGAAGAAGCTGACCCGAGCGCAGGAGAAGGTCCTGCAGAAACTGG
GGAAGGCGGACGAGACGAAGGACGAGCAGTTTGAGCAGTGTGTCCAGAAC
TTCAATAAGCAGCTGACAGAGGGTACCCGGCTGCAGAAGGATCTTCGGAC
CTATCTGGCTTCTGTTAAAGCGATGCACGAAGCCTCCAAGAAGCTGAGTG
AGTGTCTTCAGGAGGTGTATGAGCCCGAGTGGCCTGGCAGGGATGAAGCA
AACAAGATTGCAGAGAACAATGACCTACTCTGGATGGACTACCACCAGAA
GCTGGTGGACCAGGCTCTGCTGACCATGGACACCTACCTAGGCCAGTTCC
CTGATATCAAGTCGCGCATTGCCAAGCGGGGGCGGAAGCTGGTGGACTAT
GACAGTGCCCGGCACCACTATGAGTCTCTTCAAACCGCCAAAAAGAAGGA
TGAAGCCAAAATTGCCAAGGCAGAAGAGGAGCTCATCAAAGCCCAGAAGG
TGTTCGAGGAGATGAACGTGGATCTGCAGGAGGAGCTGCCATCCCTGTGG
AACAGCCGTGTAGGTTTCTATGTCAACACGTTCCAGAGCATCGCGGGTCT
GGAGGAAAACTTCCATAAAGAGATGAGTAAGCTCAATCAGAACCTCAATG
ATGTCCTGGTCAGCCTAGAGAAGCAGCACGGGAGCAACACCTTCACAGTC
AAGGCCCAACCCAGTGACAATGCCCCTGAGAAAGGGAACAAGAGCCCGTC
ACCTCCTCCAGATGGCTCCCCTGCTGCTACCCCTGAGATCAGAGTGAACC
ATGAGCCAGAGCCGGCCAGTGGGGCCTCACCCGGGGCTACCATCCCCAAG
TCCCCATCTCAGCTCCGGAAAGGCCCACCTGTCCCTCCGCCTCCCAAACA
CACCCCATCCAAGGAGATGAAGCAGGAGCAGATTCTCAGCCTTTTTGATG
ACGCATTTGTCCCTGAGATCAGCGTGACCACCCCCTCCCAGCCAGCAGAG
GCCTCCGAGGTGGTGGGTGGAGCCCAGGAGCCAGGGGAGACAGCAGCCAG
TGAAGCAACCTCCAGCTCTCTTCCGGCTGTGGTGGTGGAGACCTTCTCCG
CAACTGTGAATGGGGCGGTGGAGGGCAGCGCTGGGACTGGACGCTTGGAC
CTGCCCCCGGGATTCATGTTCAAGGTTCAAGCCCAGCATGATTACACGGC
CACTGACACTGATGAGCTGCAACTCAAAGCTGGCGATGTGGTGTTGGTGA
TTCCTTTCCAGAACCCAGAGGAGCAGGATGAAGGCTGGCTCATGGGTGTG
AAGGAGAGCGACTGGAATCAGCACAAGGAACTGGAGAAATGCCGCGGCGT
CTTCCCGGAGAATTTTACAGAGCGGGTGCAGTGA
Underlined - Exon 13
Bolded - Exon 17

In another nonlimiting exemplary embodiment, the cBIN1 polynucleotide sequence encoding a cBIN1 polypeptide or functional variant or fragment thereof is the human cBIN1 (BIN1+exon 13+exon 17) coding sequence (SEQ ID NO: 2):

Human cBIN1 Polynucleotide Sequence
SEQ ID NO: 2
ATGGCAGAGATGGGCAGTAAAGGGGTGACGGCGGGAAAGATCGCCAGCAA
CGTGCAGAAGAAGCTCACCCGCGCGCAGGAGAAGGTTCTCCAGAAGCTGG
GGAAGGCAGATGAGACCAAGGATGAGCAGTTTGAGCAGTGCGTCCAGAAT
TTCAACAAGCAGCTGACGGAGGGCACCCGGCTGCAGAAGGATCTCCGGAC
CTACCTGGCCTCCGTCAAAGCCATGCACGAGGCTTCCAAGAAGCTGAATG
AGTGTCTGCAGGAGGTGTATGAGCCCGATTGGCCCGGCAGGGATGAGGCA
AACAAGATCGCAGAGAACAACGACCTGCTGTGGATGGATTACCACCAGAA
GCTGGTGGACCAGGCGCTGCTGACCATGGACACGTACCTGGGCCAGTTCC
CCGACATCAAGTCACGCATTGCCAAGCGGGGGCGCAAGCTGGTGGACTAC
GACAGTGCCCGGCACCACTACGAGTCCCTTCAAACTGCCAAAAAGAAGGA
TGAAGCCAAAATTGCCAAGGCCGAGGAGGAGCTCATCAAAGCCCAGAAGG
TGTTTGAGGAGATGAATGTGGATCTGCAGGAGGAGCTGCCGTCCCTGTGG
AACAGCCGCGTAGGTTTCTACGTCAACACGTTCCAGAGCATCGCGGGCCT
GGAGGAAAACTTCCACAAGGAGATGAGCAAGCTCAACCAGAACCTCAATG
ATGTGCTGGTCGGCCTGGAGAAGCAACACGGGAGCAACACCTTCACGGTC
AAGGCCCAGCCCAGTGACAACGCGCCTGCAAAAGGGAACAAGAGCCCTTC
GCCTCCAGATGGCTCCCCTGCCGCCACCCCCGAGATCAGAGTCAACCACG
AGCCAGAGCCGGCCGGCGGGGCCACGCCCGGGGCCACCCTCCCCAAGTCC
CCATCTCAGCTCCGGAAAGGCCCACCAGTCCCTCCGCCTCCCAAACACAC
CCCGTCCAAGGAAGTCAAGCAGGAGCAGATCCTCAGCCTGTTTGAGGACA
CGTTTGTCCCTGAGATCAGCGTGACCACCCCCTCCCAGCCAGCAGAGGCC
TCGGAGGTGGCGGGTGGGACCCAACCTGCGGCTGGAGCCCAGGAGCCAGG
GGAGACGGCGGCAAGTGAAGCAGCCTCCAGCTCTCTTCCTGCTGTCGTGG
TGGAGACCTTCCCAGCAACTGTGAATGGCACCGTGGAGGGCGGCAGTGGG
GCCGGGCGCTTGGACCTGCCCCCAGGTTTCATGTTCAAGGTACAGGCCCA
GCACGACTACACGGCCACTGACACAGACGAGCTGCAGCTCAAGGCTGGTG
ATGTGGTGCTGGTGATCCCCTTCCAGAACCCTGAAGAGCAGGATGAAGGC
TGGCTCATGGGCGTGAAGGAGAGCGACTGGAACCAGCACAAGGAGCTGGA
GAAGTGCCGTGGCGTCTTCCCCGAGAACTTCACTGAGAGGGTCCCATGA
Underlined - Exon 13
Bolded - Exon 17

In various embodiments, the cBIN1 gene expression vector comprising a cBIN1 polynucleotide sequence encoding a cBIN1 polypeptide or functional variant or fragment thereof can be modified for better expression, production, storage, administration, detection, delivery efficiency, etc. In various embodiments, the cBIN1 gene expression vector may comprise one or more molecular tags and/or linkers. In one embodiment, the cBIN1 gene expression vector may comprise a cBIN1 polynucleotide sequence encoding one or more fluorescent tags (e.g., GFP). In another embodiment, the cBIN1 gene expression vector may comprise a cBIN1 polynucleotide sequence encoding a C-terminal or N-terminal V5 epitope tag sequence (SEQ ID NO: 3):

V5 Epitope Tag Polynucleotide Sequence
SEQ ID NO: 3
TACCCAGCTTTCTTGTACAAAGTGGTTGATCTAGAGGGCCCGCGGTTCGA
AGGTAAGCCTATCCCTAACCCTCTCCTCGGTCTCGATTCTACGCGTACCG
GTTAGTAATGA

In one nonlimiting exemplary embodiment, the cBIN1 polynucleotide sequence encoding a cBIN1 polypeptide or functional variant or fragment thereof is the mouse cBIN1 coding sequence having a C-terminal V5 epitope tag (SEQ ID NO: 4):

Mouse cBIN1 with C-terminal V5 Epitope Tag
SEQ ID NO: 4
ATGGCAGAGATGGGGAGCAAGGGGGTGACGGCGGGGAAGATCGCCAGCAA
CGTACAGAAGAAGCTGACCCGAGCGCAGGAGAAGGTCCTGCAGAAACTGG
GGAAGGCGGACGAGACGAAGGACGAGCAGTTTGAGCAGTGTGTCCAGAAC
TTCAATAAGCAGCTGACAGAGGGTACCCGGCTGCAGAAGGATCTTCGGAC
CTATCTGGCTTCTGTTAAAGCGATGCACGAAGCCTCCAAGAAGCTGAGTG
AGTGTCTTCAGGAGGTGTATGAGCCCGAGTGGCCTGGCAGGGATGAAGCA
AACAAGATTGCAGAGAACAATGACCTACTCTGGATGGACTACCACCAGAA
GCTGGTGGACCAGGCTCTGCTGACCATGGACACCTACCTAGGCCAGTTCC
CTGATATCAAGTCGCGCATTGCCAAGCGGGGGCGGAAGCTGGTGGACTAT
GACAGTGCCCGGCACCACTATGAGTCTCTTCAAACCGCCAAAAAGAAGGA
TGAAGCCAAAATTGCCAAGGCAGAAGAGGAGCTCATCAAAGCCCAGAAGG
TGTTCGAGGAGATGAACGTGGATCTGCAGGAGGAGCTGCCATCCCTGTGG
AACAGCCGTGTAGGTTTCTATGTCAACACGTTCCAGAGCATCGCGGGTCT
GGAGGAAAACTTCCATAAAGAGATGAGTAAGCTCAATCAGAACCTCAATG
ATGTCCTGGTCAGCCTAGAGAAGCAGCACGGGAGCAACACCTTCACAGTC
AAGGCCCAACCCAGTGACAATGCCCCTGAGAAAGGGAACAAGAGCCCGTC
ACCTCCTCCAGATGGCTCCCCTGCTGCTACCCCTGAGATCAGAGTGAACC
ATGAGCCAGAGCCGGCCAGTGGGGCCTCACCCGGGGCTACCATCCCCAAG
TCCCCATCTCAGCTCCGGAAAGGCCCACCTGTCCCTCCGCCTCCCAAACA
CACCCCATCCAAGGAGATGAAGCAGGAGCAGATTCTCAGCCTTTTTGATG
ACGCATTTGTCCCTGAGATCAGCGTGACCACCCCCTCCCAGCCAGCAGAG
GCCTCCGAGGTGGTGGGTGGAGCCCAGGAGCCAGGGGAGACAGCAGCCAG
TGAAGCAACCTCCAGCTCTCTTCCGGCTGTGGTGGTGGAGACCTTCTCCG
CAACTGTGAATGGGGCGGTGGAGGGCAGCGCTGGGACTGGACGCTTGGAC
CTGCCCCCGGGATTCATGTTCAAGGTTCAAGCCCAGCATGATTACACGGC
CACTGACACTGATGAGCTGCAACTCAAAGCTGGCGATGTGGTGTTGGTGA
TTCCTTTCCAGAACCCAGAGGAGCAGGATGAAGGCTGGCTCATGGGTGTG
AAGGAGAGCGACTGGAATCAGCACAAGGAACTGGAGAAATGCCGCGGCGT
CTTCCCGGAGAATTTTACAGAGCGGGTGCAGTACCCAGCTTTCTTGTACA
AAGTGGTTGATCTAGAGGGCCCGCGGTTCGAAGGTAAGCCTATCCCTAAC
CCTCTCCTCGGTCTCGATTCTACGCGTACCGGTTAGTAATGA
Underlined - Exon 13
Bolded - Exon 17
Italicized - V5 Tag

In various embodiments, the cBIN1 polynucleotide sequence encoding a cBIN1 polypeptide or functional variant or fragment thereof may comprise one or more mutations. For example, in various embodiments, the cBIN1 polynucleotide sequence encodes cBIN1 polypeptide functional variants having one or more conservative amino acid substitutions, where the variant retains a substantial amount of biological activity. Exemplary conservative amino acid substitution groups include, but are not limited to, valine-leucine-isoleucine, phenylalanine-tyrosine, lysine-arginine, alanine-valine, aspartic acid-glutamic acid, and asparagine-glutamine.

In some embodiments, the cBIN1 polynucleotide sequence encoding a cBIN1 polypeptide or functional variant or fragment thereof comprises one or more mutations and has at least about 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence homology to the wildtype cBIN1 gene while still retaining a substantial amount of biological activity. For example, the cBIN1 polynucleotide sequence may have greater than about 90%, greater than about 95%, or greater than about 99% sequence homology to the wildtype cBIN1 gene while still retaining a substantial amount of biological activity.

### Methods of Treatment

The present disclosure also provides methods of treating, preventing, reducing the likelihood of having, reducing the severity of, and/or slowing the progression of DCM in a subject.

The methods may comprise administering to the subject a therapeutically effective amount of a pharmaceutical composition comprising: a cBIN1 gene expression vector comprising a cBIN1 polynucleotide sequence encoding a cBIN1 polypeptide or functional variant or fragment thereof, as described herein. In some embodiments, the subject may have type 1 diabetes or type 2 diabetes. In some embodiments, the subject may have DCM, or may be at risk of developing DCM.

In various embodiments, the pharmaceutical composition comprising the cBIN1 gene expression vector comprising a cBIN1 polynucleotide sequence encoding a cBIN1 polypeptide or functional variant or fragment thereof may be administered as a therapeutic agent (e.g., cBIN1 gene therapy) to a subject to treat, prevent, reduce the likelihood of having, reduce the severity of, and/or slow the progression of DCM in the subject. In various embodiments, the pharmaceutical composition may be administered as a therapeutic agent (e.g., cBIN1 gene therapy) to a subject to reduce blood glucose levels in the subject.

In various embodiments, the subject is a mammal. In various embodiments, the subject is a human. In various embodiments, the subject is a mammalian subject including but not limited to human, monkey, ape, dog, cat, cow, horse, goat, pig, rabbit, mouse, and rat.

In various embodiments, the pharmaceutical composition comprising a cBIN1 gene expression vector comprising a cBIN1 polynucleotide sequence encoding a cBIN1 polypeptide or functional variant or fragment thereof is administered to a subject by intravenous (i.v.) injection. Pharmaceutical compositions as described herein may also be administered using alternative routes, including but not limited to intravascular, intraarterial, intramuscular, subcutaneous, intraperitoneal, aerosol, nasal, via inhalation, oral, transmucosal, transdermal, parenteral, implantable pump or reservoir, continuous infusion, enteral application, topical application, local application, capsules, and/or injections. For example, in one embodiment, the pharmaceutical composition comprising a cBIN1 gene expression vector comprising a cBIN1 polynucleotide sequence encoding a cBIN1 polypeptide or functional variant or fragment thereof is administered to a subject by cardiac catheter infusion to myocardium.

In some embodiments, the subject is administered a single dose of the disclosed pharmaceutical compositions. In other embodiments, the subject is administered a plurality of doses of the disclosed pharmaceutical compositions over a period of time. For example, in various nonlimiting embodiments, a pharmaceutical composition as described herein may be administered to a subject once a day (SID/QD), twice a day (BID), three times a day (TID), four times a day (QID), or more, so as to administer a therapeutically effective amount of the pharmaceutical composition to the subject, where the therapeutically effective amount is any one or more of the doses described herein. In some embodiments, a pharmaceutical composition as described herein is administered to a subject 1-3 times per day, 1-7 times per week, 1-9 times per month, 1-12 times per year, or more. In other embodiments, a pharmaceutical composition as described herein is administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a pharmaceutical composition as described herein is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, 900-1000 mg/kg, or a combination thereof.

The actual dosing regimen can depend upon many factors, including but not limited to the judgment of a trained physician, the overall condition of the subject, and the specific type of DCM. The actual dosage can also depend on the determined experimental effectiveness of the specific pharmaceutical composition (e.g., a cBIN1 gene expression vector comprising a cBIN1 polynucleotide sequence encoding a cBIN1 polypeptide or functional variant or fragment thereof) that is administered. For example, the dosage may be determined based on in vitro responsiveness of relevant cultured cells, or in vivo responses observed in appropriate animal models or human studies for the cBIN1 gene therapy.

In some embodiments, disclosed pharmaceutical compositions may be administered to a subject using a dosing regimen that is based on cBIN1 gene expression vector genome (vg) per kg body weight of the subject (vg/kg). In one embodiment, a cBIN1 gene therapy vg/kg dosing regimen is used to treat DCM in a subject. In another embodiment, a cBIN1 gene therapy vg/kg dosing regimen is used to reduce blood glucose levels in a subject. In another embodiment, a cBIN1 gene therapy vg/kg dosing regimen is used to treat DCM and reduce blood glucose levels in a subject. In one nonlimiting exemplary embodiment, a pharmaceutical composition comprising a cBIN1 gene therapy is administered to a subject at a dose ranging from about 5×10¹¹ vg/kg to about 5×10¹³ vg/kg cBIN1 gene expression vector.

In some embodiments, the disclosed methods and pharmaceutical compositions are able to sufficiently deliver a cBIN1 transgene to a large mammalian (e.g., human) heart by i.v. administration of a very low dose of cBIN1 gene therapy (e.g., about 5×10¹¹ vg/kg to about 5×10¹³ vg/kg cBIN1 gene expression vector). In some embodiments, the disclosed methods and pharmaceutical compositions are able to achieve robust and durable cBIN1 transgene expression in a large mammalian heart, specifically in the apex, anterior wall, posterior wall, base, and/or septum regions of the heart. In some embodiments, the disclosed methods and pharmaceutical compositions are able to increase the selective expression of a cBIN1 transgene in a large mammalian heart by at least about 20% relative to an endogenous cBIN1 expression level in the heart. In some embodiments, the disclosed methods and pharmaceutical compositions are able to increase the selective expression of a cBIN1 transgene in a large mammalian heart for at least 6 months.

Another embodiment described herein is the use of echocardiography or other imaging methods to measure cardiac and functional improvements of a subject in response to treatment with the disclosed cBIN1 gene therapies.

Another embodiment described herein is the use of a plasma or blood cardiac bridging integrator 1 (cBIN1) score (CS) to identify a subject having diabetic cardiomyopathy for cBIN1 gene therapy treatment. In one aspect, the cBIN1 gene therapy treatment comprises a therapeutically effective amount of a pharmaceutical composition comprising: a cBIN1 gene expression vector comprising a cBIN1 polynucleotide sequence encoding a cBIN1 polypeptide or functional variant or fragment thereof. In another aspect, the plasma CS is a non-invasive measure of target engagement and therapeutic response of the subject to the cBIN1 gene therapy treatment. In another aspect, the plasma CS is the natural log of the ratio of a median plasma cBIN1 concentration in a normal human population to a measured cBIN1 concentration in the subject having diabetic cardiomyopathy.

In some embodiments, the disclosed methods and pharmaceutical compositions may reduce blood glucose levels in a subject. In some embodiments, the disclosed methods and pharmaceutical compositions may normalize cardiac glucose uptake in a subject. In some embodiments, the disclosed methods and pharmaceutical compositions may stabilize (i.e., normalize or restore) the intracellular distribution of calcium handling machinery in myocardium of a subject, wherein the calcium handling machinery may comprise one or more of SERCA2a, Cav1.2, or RyR₂. In some embodiments, the disclosed methods and pharmaceutical compositions may rehabilitate or increase transverse-tubule microfolds or microdomains in myocardium of a subject. In some embodiments, the disclosed methods and pharmaceutical compositions may normalize/increase the localization and/or trafficking of GLUT4 to the cardiac T-tubule. In some embodiments, the disclosed methods and pharmaceutical compositions may correct T-tubule muscle pathology present in a diabetic heart and/or improve, restore, or enhance T-tubule size, shape, and/or structure, as measured by a tissue biopsy or other means for imaging cardiac T-tubules.

In some embodiments, the disclosed methods and pharmaceutical compositions may selectively express cBIN1 in the heart of a subject. In some embodiments, the disclosed methods and pharmaceutical compositions may selectively express cBIN1 in one or more of the apex, anterior wall, posterior wall, base, or septum of the heart of a subject. In some embodiments, the disclosed methods and pharmaceutical compositions may increase the selective expression of cBIN1 in the heart of a subject by at least about 20% relative to an endogenous cBIN1 expression level in the heart of the subject. In some embodiments, the disclosed methods and pharmaceutical compositions may normalize cardiac expression of cBIN1 in a subject having a diabetic heart. In some embodiments, the disclosed methods and pharmaceutical compositions may express cBIN1 in a subject for at least 6 months. In some embodiments, the disclosed methods and pharmaceutical compositions may improve exercise tolerance and/or functional capacity in a subject having, or at risk of developing, diabetic cardiomyopathy. In some embodiments, the disclosed methods and pharmaceutical compositions may enhance and/or normalize the cardiac expression of cBIN1 to a sufficient level to halt, prevent, or reverse heart failure in a subject having, or at risk of developing, diabetic cardiomyopathy. In some embodiments, the disclosed methods and pharmaceutical compositions may reverse diastolic dysfunction (including normalization of end diastolic volume, E/A, E/e′, and/or isovolumic relaxation time) and improve systolic function (including cardiac output) in a subject having, or at risk of developing, diabetic cardiomyopathy.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method of treating, preventing, reducing the likelihood of having, reducing the severity of, and/or slowing the progression of diabetic cardiomyopathy in a subject, the method comprising:
   administering to the subject a therapeutically effective amount of a pharmaceutical composition comprising:
      a cardiac bridging integrator 1 (cBIN1) gene expression vector comprising a cBIN1 polynucleotide sequence encoding a cBIN1 polypeptide or functional variant or fragment thereof.
Clause 2. The method of clause 1, wherein the pharmaceutical composition is administered to the subject by intravenous (i.v.) injection.
Clause 3. The method of clause 1 or 2, wherein the pharmaceutical composition is administered to the subject by cardiac catheter infusion to myocardium.
Clause 4. The method of any one of clauses 1-3, wherein the cBIN1 polynucleotide sequence comprises DNA, RNA, or a combination thereof.
Clause 5. The method of any one of clauses 1-4, wherein the cBIN1 polynucleotide sequence has at least 90-99% identity to any one of SEQ ID NO: 1, 2, or 4.
Clause 6. The method of any one of clauses 1-5, wherein the cBIN1 polynucleotide sequence is any one of SEQ ID NO: 1, 2, or 4.
Clause 7. The method of any one of clauses 1-6, wherein the cBIN1 gene expression vector is selected from a non-viral vector, a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.
Clause 8. The method of any one of clauses 1-7, wherein the cBIN1 gene expression vector is an AAV vector of a serotype selected from AAV1, AAV2, AAV3, AAV4, AAV5, AAV6, AAV7, AAV8, AAV9, AAV10, AAV11, AAV12, AAVrh74, a hybrid serotype thereof, or a derivative thereof.
Clause 9. The method of any one of clauses 1-8, wherein the cBIN1 gene expression vector is an AAV9 vector.
Clause 10. The method of any one of clauses 1-9, wherein the cBIN1 gene expression vector is a muscle-tropic AAV vector.
Clause 11. The method of any one of clauses 1-10, wherein the non-viral vector comprises a lipid carrier, an exosome, a polymer-based carrier, a chemical-based carrier, a conjugated carrier, or combinations thereof.
Clause 12. The method of any one of clauses 1-11, wherein the pharmaceutical composition is administered to the subject using a dosing regimen based on cBIN1 gene expression vector genome (vg) per kg body weight of the subject.
Clause 13. The method of any one of clauses 1-12, wherein the pharmaceutical composition is administered to the subject at a dose ranging from about 5×10¹¹ vg/kg to about 5×10¹³ vg/kg cBIN1 gene expression vector.
Clause 14. The method of any one of clauses 1-13, wherein the subject has type 1 diabetes or type 2 diabetes.
Clause 15. The method of any one of clauses 1-14, wherein the subject has diabetic cardiomyopathy.
Clause 16. The method of any one of clauses 1-15, wherein the pharmaceutical composition reduces blood glucose levels in the subject.
Clause 17. The method of any one of clauses 1-16, wherein the pharmaceutical composition normalizes or restores the intracellular distribution of calcium handling machinery in myocardium of the subject, wherein the calcium handling machinery comprises one or more of SERCA2a, Cav1.2, or RyR₂.
Clause 18. The method of any one of clauses 1-17, wherein the pharmaceutical composition rehabilitates or increases transverse-tubule microfolds or microdomains in myocardium of the subject.
Clause 19. The method of any one of clauses 1-18, wherein the pharmaceutical composition selectively expresses the cBIN1 polypeptide or functional variant or fragment thereof in the heart of the subject.
Clause 20. The method of any one of clauses 1-19, wherein the pharmaceutical composition increases selective expression of the cBIN1 polypeptide or functional variant or fragment thereof in the heart of the subject by at least about 20% relative to an endogenous cBIN1 expression level in the heart of the subject.
Clause 21. The method of any one of clauses 1-20, wherein the pharmaceutical composition expresses the cBIN1 polypeptide or functional variant or fragment thereof in the subject for at least 6 months.
Clause 22. The method of any one of clauses 1-21, wherein the subject is a mammal.
Clause 23. The method of any one of clauses 1-22, wherein the subject is a human.
Clause 24. A pharmaceutical composition comprising a cardiac bridging integrator 1 (cBIN1) gene expression vector comprising a cBIN1 polynucleotide sequence encoding a cBIN1 polypeptide or functional variant or fragment thereof.
Clause 25. The pharmaceutical composition of clause 24, wherein the cBIN1 polynucleotide sequence comprises DNA, RNA, or a combination thereof.
Clause 26. The pharmaceutical composition of clause 24 or 25, wherein the cBIN1 polynucleotide sequence has at least 90-99% identity to any one of SEQ ID NO: 1, 2, or 4.
Clause 27. The pharmaceutical composition of any one of clauses 24-26, wherein the cBIN1 polynucleotide sequence is any one of SEQ ID NO: 1, 2, or 4.
Clause 28. The pharmaceutical composition of any one of clauses 24-27, wherein the cBIN1 gene expression vector is selected from a non-viral vector, a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.
Clause 29. The pharmaceutical composition of any one of clauses 24-28, wherein the cBIN1 gene expression vector is an AAV vector of a serotype selected from AAV1, AAV2, AAV3, AAV4, AAV5, AAV6, AAV7, AAV8, AAV9, AAV10, AAV11, AAV12, AAVrh74, a hybrid serotype thereof, or a derivative thereof.
Clause 30. The pharmaceutical composition of any one of clauses 24-29, wherein the cBIN1 gene expression vector is an AAV9 vector.
Clause 31. The pharmaceutical composition of any one of clauses 24-30, wherein the cBIN1 gene expression vector is a muscle-tropic AAV vector.
Clause 32. The pharmaceutical composition of any one of clauses 24-31, wherein the non-viral vector comprises a lipid carrier, an exosome, a polymer-based carrier, a chemical-based carrier, a conjugated carrier, or combinations thereof.
Clause 33. Use of a cardiac bridging integrator 1 (cBIN1) gene therapy in a medicament for treating, preventing, reducing the likelihood of having, reducing the severity of, and/or slowing the progression of diabetic cardiomyopathy in a subject.
Clause 34. The use of clause 33, wherein the cBIN1 gene therapy comprises a therapeutically effective amount of a pharmaceutical composition comprising:

a cBIN1 gene expression vector comprising a cBIN1 polynucleotide sequence encoding a cBIN1 polypeptide or functional variant or fragment thereof.
Clause 35. Use of a plasma cardiac bridging integrator 1 (cBIN1) score (CS) to identify a subject having diabetic cardiomyopathy for cBIN1 gene therapy treatment.
Clause 36. The use of clause 35, wherein the cBIN1 gene therapy treatment comprises a therapeutically effective amount of a pharmaceutical composition comprising:

a cBIN1 gene expression vector comprising a cBIN1 polynucleotide sequence encoding a cBIN1 polypeptide or functional variant or fragment thereof.
Clause 37. The use of clause 35 or 36, wherein the plasma CS is a non-invasive measure of target engagement and therapeutic response of the subject to the cBIN1 gene therapy treatment.
Clause 38. The use of any one of clauses 35-37, wherein the plasma CS is the natural log of the ratio of a median plasma cBIN1 concentration in a normal human population to a measured cBIN1 concentration in the subject having diabetic cardiomyopathy.

### EXAMPLES

### Example 1

### Materials and Methods

### Animal Studies

An equal amount of male and female C57BLKS/J genetic background BKS.Cg-Dock7^{m}+/+Lepr^{db}/J mice (The Jackson Laboratory) were used for this study. Mice homozygous for the spontaneous point mutation in the gene encoding the leptin receptor Lepr^{db} (db/db) manifest metabolic abnormalities including obesity, dyslipidemia, and type 2 diabetes mellitus (T2DM). The lean heterozygous Dock7^{m}+/+Lepr^{db} littermates (db/m) served as the control group, which can be identified from homozygotes before the phenotype becomes severe. In addition, adult male and female mice with cardiac-specific Bin1 heterozygote (HT) deletion (Bin1^{fl/+}; Myh6-Cre⁺) with their WT (Bin1^{+/+}; Myh6-Cre⁺) littermates were used. Mice were kept at less than 5 mice/cage in temperature-controlled cages (20-22° C.) with a 12:12-hr light: dark cycle and free access to water and food. For the minipig study, 5- to 7-month-old male and female Yucatan minipigs were purchased from S & S Farms and housed in their housing pen.

### Adult Primary Cardiomyocyte Isolation and Culture

Adult mouse cardiomyocytes (CMCs) were isolated from both male and female mice with collagenase II (2 mg/mL, Worthington Biochemical Corporation) using a previously described method. Freshly isolated CMCs were seeded in laminin-precoated culture dishes and cultured in perfusion buffer (120.4 mM NaCl, 14.7 mM KCl, 0.6 mM KH₂PO₄, 0.6 mM Na₂HPO₄, 1.2 mM MgSO₄·7H₂O, 10 mM HEPES, 4.6 mM NaHCO₃, 30 mM taurine, and 5.5 mM glucose) for 1 hr in 37° C. and 5% CO₂ incubator prior to further experiments.

### Generation and Administration of Adeno-Associated Virus 9 (AAV9)

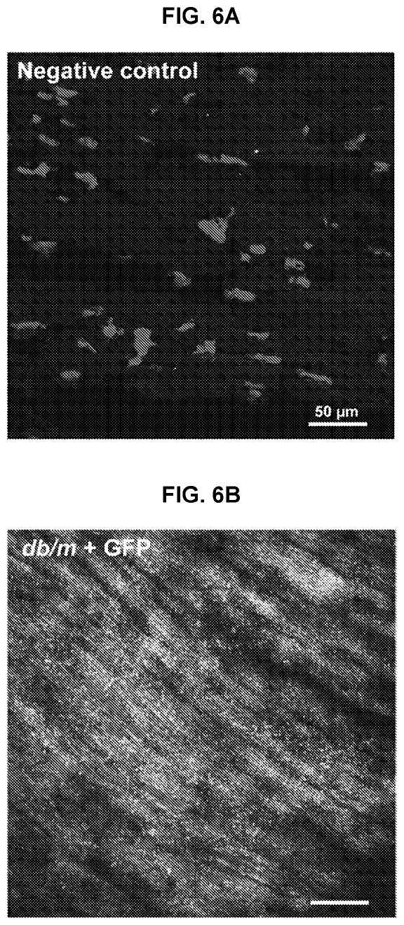

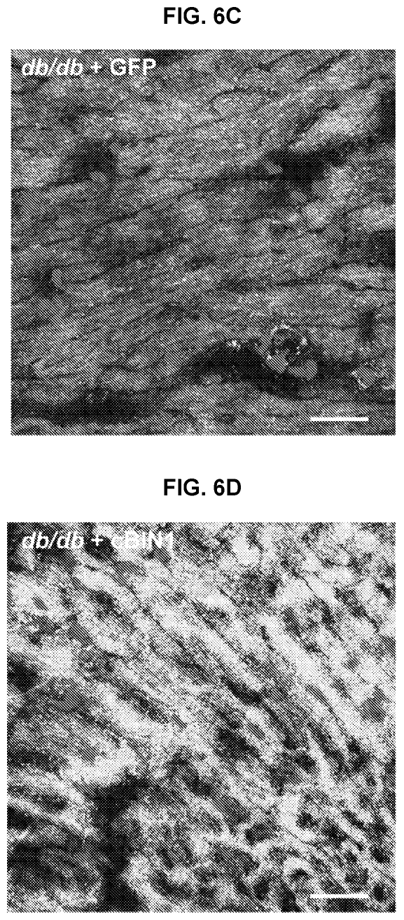

Nine-week-old male and female db/db mice and littermate control db/m mice were randomized to receive 100 μL of 1×10¹¹ vector genome (vg) of AAV9 transducing V5-tagged GFP or cBIN1 (SEQ ID NO: 4) (n=16-17 per group) or an equal volume (100 μL) of PBS (n=5 per group) via retro-orbital injection after anesthesia (1% isoflurane in oxygen). Eight weeks after injection, 17-week-old mice were terminated for further experiments. The rationale to use AAV9-CMV vectors for cardiac tropism expression, GFP as a negative control virus, viral administration route, and the detailed viral preparation method (custom produced at Welgen, Inc.) were previously described in detail (Liu et al., JACC Basic Trans/Sci., 5:561-578 (2020)). To increase the cardiomyocyte transduction efficacy, the viral dose was increased from 3×10¹⁰ vg/animal, which previously induced transduction in around 60% of cardiomyocytes, to a dose of 1×10¹¹ vg/animal, which has previously been reported to be effective in transducing 81.2% of cardiomyocytes with limited transduction in liver (5.3%) and skeletal muscle (3.2%). Using anti-V5 labeling for exogenous proteins (GFP-V5 or cBIN1-V5), it was identified that a single retro-orbital administration of 1×10¹¹ vg/animal AAV9-CMV-GFP or cBIN1 (SEQ ID NO: 4) was able to transduce 100% of cardiomyocytes at 8 weeks following viral injection (FIG. 6).

A similar protocol was repeated in older db/m or db/db mice at 6 months of age that were injected with AAV9-CMV-GFP/cBIN1 (1×10¹¹ vg, retro-orbital injection) and evaluated for echocardiography parameters and treadmill performance 8 weeks later at 8 months of age. The mice were then terminated. In addition to the CMV promoter, an AAV9-cBIN 1/GFP-V5 using an AAV9 vector driven under the previously established cardiac-specific promoter cTnT was also created. The treatment protocol, dosage, and administration route of AAV9-cTnT-cBIN1/GFP-V5 remained the same as those used for the AAV-CMV viruses. In brief, 9-week-old male and female db/m and db/db mice received a dose of 1×10¹¹ vg/animal AAV9-cTnT-GFP or cBIN1-V5 via retro-orbital injection (n=12 mice per group) and were terminated 8 weeks later at 17 weeks old. The same AAV9 injection protocol was used for studies involving a cardiac-specific Bin1 HT mouse line. In brief, 13-month-old male and female Bin1 HT mice and their WT littermate controls were administered with AAV9-CMV-cBIN1 (1×10¹¹ vg) and were terminated 8 weeks later for i.p. glucose tolerance test (iGTT) and i.p. insulin tolerance test (iITT) before being subjected to cardiomyocyte isolation for imaging and functional analysis.

For minipig studies, a single dose of i.v. injected AAV9-CMV-cBIN1-V5 (6×10¹¹ vg/kg) was administered to minipigs via an ear vein injection method. Six months after injection, animals were terminated and heart tissue was obtained for mRNA extraction and cDNA preparation.

### Experimental Protocol

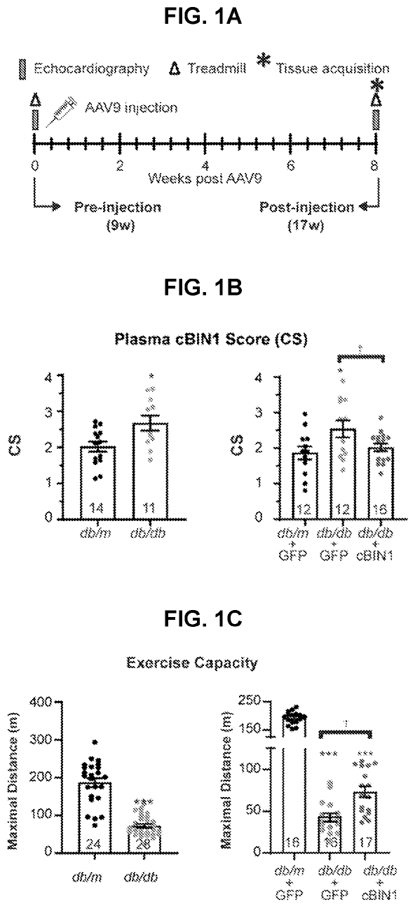

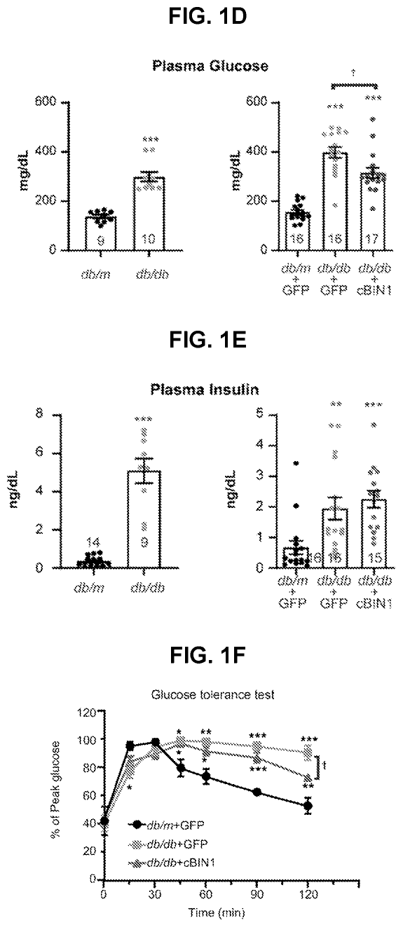

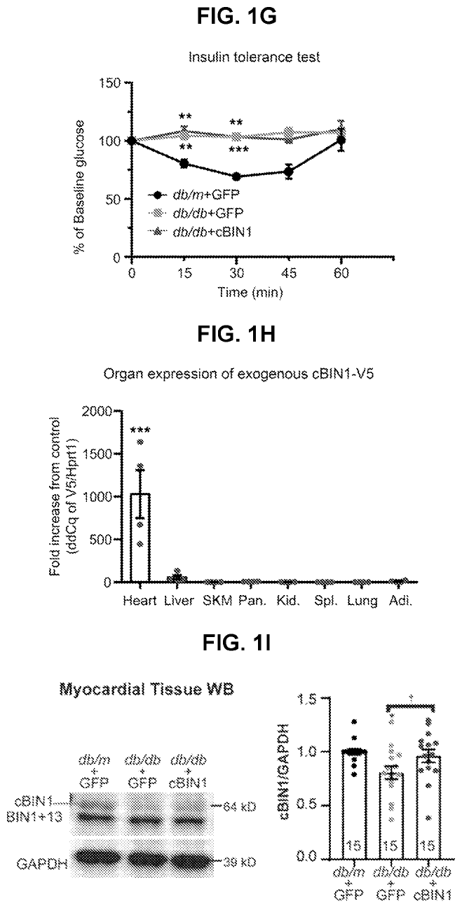

All mice receiving AAV9 were terminated 8 weeks post viral injection at 17-weeks of age (FIG. 1A). Echocardiography, plasma CS values, and exercise tolerance were obtained before and 8-weeks after AAV9 injection. Intraperitoneal glucose/insulin tolerance tests were performed in fasting mice prior to termination. Gross physiological parameters including body weight, heart and lung weight, and tibia length were obtained at termination. Hearts were processed for transmission electron microscopy imaging, biochemical analysis including t-tubule fractionation, or mass-spectrometry analysis. Ventricular cardiomyocytes or mitochondria were isolated for cardiomyocyte glucose uptake assay, immunofluorescence labeling and spinning-disc confocal imaging, seahorse analysis of cell respiration, or isolated mitochondrial oxidative stress analyzed by Oroboros respirometers and mitochondrial calcium retention analyzed by Horiba fluorometers.

### Echocardiography

In vivo echocardiography was obtained using a Vevo-3100 ultrasound system (Visual Sonics). M-mode images in LV short axis view at the proximal level of papillary muscles were used for measurement of LV internal diameter, anterior and posterior wall thickness, LV mass, and relative wall thickness. Parasternal LV long axis view of two-dimensional image was used for measurement of volume related parameters using LV trace method followed by automatic calculation by the Vevo Software (Visual Sonics). Analysis of 3 to 5 cardiac cycles was used to generate average result for each parameter. Doppler echocardiography in apical four-chamber view was obtained for diastolic functional analysis. Transmittal flow velocity (E, A) was obtained by pulse-wave Doppler placing the sample volume at the mitral leaflet tips. Wall velocity (e′) was obtained by Tissue Doppler placing the sample volume at the septal mitral annulus.

### Exercise Tolerance Test

Exercise capacity was estimated using db/m and db/db mice before and after AAV9 treatment at 9 weeks and 17 weeks, respectively. This test was completed to unmask cardiac abnormalities that might not be sufficient to limit oxygen supply-demand relationships at rest, but that could prevent oxygen delivery from meeting heightened myocardial oxygen demand during dynamic exercise (e.g., treadmill-running).

The current exercise tolerance test followed an established protocol with minor adjustments. To familiarize the mice with treadmill-running, the mice being tested ran on the treadmill (Columbus Instruments) at 20° incline×5 m/min for 10 min per day×3 days. At least 24 hr following the final acclimation period, mice ran 20° incline×5 m/min for 6 min, 7 m/min for 3 min, 9 m/min for 3 min, 11 m/min for 3 min, 13 m/min for 3 min, 15 m/min for 3 min, and 17 m/min until the animal could no longer maintain pace with the treadmill belt for >10 consecutive seconds. The first 3 min were used for animal acclimatization to the treadmill at the day of experiment, the distance during which is not included in data collection. Maximal running distance was recorded during the test.

### Transmission Electron Microscopy

After sacrifice, mouse coronary artery was retrogradely perfused through aorta with 20 mL of ice-cold fixative (formaldehyde/glutaraldehyde 2.5% in 0.1M Sodium Cacodylate Buffer). After perfusion, left ventricles were sliced into 1 mm³ tissue sections, stored at 4° C. in fixative, and sent for further processing. After post fixation with 2% osmium tetroxide and pre-staining with uranyl acetate, the ventricular tissue slices were dehydrated in graded ethanol and pure acetone, imbedded with epoxy resin, and sectioned at 70 nm using an ultramicrotome (Leica). Sections were poststained with acetate and lead citrate before imaging with JEM-1400 plus or JEM1200-EX (JEOL) transmission electron microscope with CCD Gatan camera. The degree of t-tubule contour was graded using a modified scoring system that was reported previously.

Plasma Collection, Component Analysis CS, and Myocardial Tissue cBIN1 Analysis

All plasma samples from mice anesthetized with isoflurane were collected from the submandibular vein with anticoagulant EDTA. Whole blood samples were centrifuged at 2,250×g for 20 min at 4° C. Plasma was separated and aliquoted before flash freezing with dry ice and ethanol. Plasma aliquots were stored at −80° C. before assays.

The plasma cBIN1 score (CS), which is the natural log of the ratio of median plasma [cBIN1] in a normal human population to the measured cBIN1, was determined following the manufacturer's instructions of the CS Quantification ELISA kit (Sarcotein Diagnostics) using the below equation provided in the kit.

$$CS = ln ( \frac{100}{[ cBIN 1 ]} )$$

Plasma levels of brain natriuretic peptide (BNP), atrial natriuretic peptide (ANP), glucose, and insulin were determined using the BNP EIA Kit (Sigma-Aldrich), the ANP EIA Kit (Sigma-Aldrich), the Mouse Glucose Assay Kit (Crystal Chem), and the Ultra-Sensitive Mouse Insulin ELISA Kit (Crystal Chem), respectively.

### Adult Primary Cardiomyocyte Glucose Uptake Test

Adult mouse cardiomyocytes were isolated from both male and female mice with collagenase II (2 mg/mL, Worthington Biochemical Corporation). Freshly isolated cardiomyocytes were seeded in laminin-precoated culture dishes and cultured in perfusion buffer (120.4 mM NaCl, 14.7 mM KCl, 0.6 mM KH₂PO₄, 0.6 mM Na₂HPO₄, 1.2 mM MgSO₄×7H₂O, 10 mM Hepes, 4.6 mM NaHCO₃, 30 mM taurine, and 5.5 mM glucose) for 1 hour in 37° C. and 5% CO₂ incubator prior to further experiments.

For glucose uptake assays, freshly isolated CMCs were seeded and cultured in laminin-precoated 96-well plates for 1 hour prior to the test. Cells were then washed twice with glucose-free perfusion buffer (120.4 mM NaCl, 14.7 mM KCl, 0.6 mM KH₂PO₄, 0.6 mM Na₂HPO₄, 1.2 mM MgSO₄: 7H₂O, 10 mM HEPES) and starved in starvation buffer (glucose-free perfusion buffer containing 1 mM pyruvate, 0.1% BSA) for 40 min. Metformin (0, 20 μM) was loaded together with starvation buffer in the indicated groups. Cells were then stimulated in the absence or presence of insulin (0, 10 nM) (Sigma-Aldrich) for 20 min. Following the manufacturer's instructions, 2 DG-glucose uptake test in CMCs was then determined by Glucose Uptake Assay kit (Abcam) and quantified with a FlexStation 3 plate reader (Molecular Devices).

[U-¹³C₆]-Glucose Tracing and Liquid Chromatography-MS (LC-MS) Analysis of Polar Metabolites

The metabolites were extracted from isolated primary cardiomyocytes using previously established methods. Briefly, the cardiomyocytes were plated on laminin-coated 6 cm dishes in biological triplicates. Cells were initially plated on standard culture medium and washed with sterile PBS. Culture medium in which glucose was replaced by [¹³C₆]-L-glucose (Cambridge Isotope Laboratories) was then added to the cells. Cells were allowed to grow in labeled media for 4 hours to reach steady state. Then, medium was rapidly aspirated (¹³C-labeled media were collected), and cells were washed with cold 0.9% saline on ice. In total, 3 mL of extraction solvent (80% methanol/water, precooled to −80° C.) was added to each well, and the dishes were transferred to −80° C. for 15 minutes. Cells were then scraped into the extraction solvent on dry ice. The supernatant of each sample was then vortexed a couple of times (30 seconds each). All metabolite extracts were centrifuged at 20,000×g at 4° C. for 10 minutes. Each sample was then transferred to a new 1.5 mL tube. Finally, the solvent in each sample was evaporated in a Speed Vacuum (Savant UVS450, Thermo Fisher Scientific) and stored at −80° C. until samples were run on a QExactive HF orbitrap mass spectrometer (Thermo Fisher Scientific). Extracted peak intensities were corrected for naturally occurring ¹³C isotope abundance before analysis.

Extracted polar metabolite samples were analyzed by LC-MS. Separation was achieved by hydrophilic interaction LC (HILIC) using a Vanquish HPLC system (Thermo Fisher Scientific). The column was an Xbridge BEH amide column (2.1 mm×150 mm, 2.5 UM particular size, 130 Å pore size, Waters Co.) run with a gradient of solvent A (20 mM ammonium hydroxide, 20 mM ammonium acetate in 95:5 acetonitrile/water, pH 9.5) and solvent B (100% acetonitrile) at a constant flow rate of 150 μL/min. The gradient function was: 0 minutes, 90% B; 2 minutes, 90% B; 3 minutes, 75% B; 7 minutes, 75% B; 8 minutes, 70% B; 9 minutes, 70% B; 10 minutes, 50% B; 12 minutes, 50% B; 13 minutes, 25% B; 14 minutes, 25% B; 16 minutes, 0% B; 20.5 minutes, 0% B; 21 minutes; 90% B; and 25 minutes, 90% B. Autosampler temperature was 4° C., column temperature was 30° C., and injection volume was 3 μL. Samples were injected by electrospray ionization into a QExactive HF orbitrap mass spectrometer (Thermo Fisher Scientific) operating in negative ion mode with a resolving power of 120,000 at m/z of 200 and a full scan range of 75-1,000 m/z. Data were analyzed using the EL-MAVEN software package, and specific peaks were assigned based on exact mass and comparison with known standards.

### Seahorse Respiration Analysis

Isolated CMC oxygen consumption rates (OCR) were measured with Seahorse XFe96 Extracellular Flux Analyzer (Agilent) using Seahorse XF Cell Mito Stress kit (Agilent) as previously described with modification. In brief, isolated adult CMCs were seeded in laminin-precoated Agilent 96-well seahorse cell culture plates (1,500 cells/well) and allowed attachment in perfusion buffer for 1 hr. The plating medium was then removed and replaced by seahorse assay medium supplemented with 5.5 mM glucose, 1 mM pyruvate, and 4 mM L-glutamine, and the cells were cultured in a non-CO₂ incubator at 37° C. for 1 hr before assay. The level of OCR/ECAR were measured before (baseline) and after the sequential stimulation of oligomycin (10 μM) for leak respiration, carbonyl cyanide p-trifluoromethoxyphenylhydrazone (FCCP, 1 μM) for maximal uncoupled respiration, and antimycin A/rotenone (1 μM) for residual respiration. Maximal and spare OCR were calculated and adjusted for cell count.

### Mitochondrial Analysis

Cardiac mitochondria were isolated according to a previously established protocol with modification. The hearts were collected and immediately minced on ice in mitochondrial isolation medium (MIM) buffer (300 mM sucrose, 10 mM HEPES, 1 mM EGTA, and 1 mg/mL BSA, pH 7.4) and homogenized using Teflon-glass system. The homogenate was centrifuged at 800×g for 10 min at 4° C., and the supernatant was re-centrifuged at 800×g for 10 min at 4° C. Then, the supernatant was centrifuged at 12,000×g for 10 min at 4° C. for the crude mitochondrial pellet, which was resuspended in MIM for experiments. Mitochondrial calcium retention and H₂O₂ production were analyzed by high-resolution O₂K oxygraphs (Oroboros) and Fluoromax 3 fluorometers (Horiba) using previously established protocols. In brief, for mitochondrial calcium retention analysis, isolated cardiac mitochondria were loaded in Ca5N (1 μM) assay buffer supplemented with 10 mM malate, 10 mM glutamate, 50 μM ADP, and calcium was added to increase matrix Ca²⁺ load. The fluorescence intensity was recorded. The mitochondrial H₂O₂ production was measured using the Amplex Ultrared (10 μM)/horseradish peroxidase (HRP, 3 U/mL) detection system. Isolated cardiac mitochondria were added to assay buffer containing Amplex Ultra Red and HRP, and a 5-min background rate was measured following the addition of succinate (10 mM) for measurement of H₂O₂ production rate. Both calcium retention capacity and H₂O₂ production rates were corrected for O₂ consumption.

### Cardiac Microsome Preparation and Sucrose Gradient Fractionation

Microsome sucrose gradient fractionation was prepared according to an established protocol with modifications. Frozen hearts were minced in 2 mL ice-cold homogenization buffer (20 mM Tris pH 7.4, 250 mM sucrose, 1 mM EDTA supplemented with HALT protease inhibitor). After homogenization with a Polytron Handheld homogenizer (low speed, 15,000 rpm), lysates were centrifuged at 12,000×g for 20 min at 4° C. and the supernatant (S1) was collected and kept on ice. The pellet was then resuspended in 1 mL of the same homogenization buffer, homogenized, and centrifuged again at 12,000×g for 20 min at 4° C. to prepare supernatant (S2). Microsomal supernatants were then combined (S1+S2) and subjected to ultracentrifugation in a fixed angle rotor Ti 50.2 at 110,000×g for 2 hr at 4° C. using the Beckman Ultracentrifuge L8-70M (Beckman Coulter). After ultracentrifugation, the pellet was weighted, resuspended in the appropriate amount of homogenization buffer (˜0.5 mL) for a final concentration of microsome of 4 mg/mL. The same amount of total microsome from each heart sample (˜1.5 mg in 0.5 mL, protein concentration determined by BCA assay) was laid over the top of the prepared sucrose gradient (2 mL for 27%, 2 mL for 32%, 2 mL for 38%, and 3 mL for 45%, v/w in homogenization buffer) and ultracentrifuged in a swinging-bucket rotor SW 28 (Beckman) at 77,000×g for 16 hr. Around 1 mL samples were collected from each of the following fractions: F1, 27%; F2, 27/32%; F3, 32/38%; and F4, 38/45%. Recovered fractions were diluted 4× in homogenization buffer and ultracentrifuged again (Beckman, Ti 50.2) at 120,000×g for 2 hr at 4° C. The microsome pellets obtained from each fraction were resuspended in 60 μL of homogenization buffer (protein concentration determined by BCA), added with sample buffer, and aliquoted and frozen at −20° C. for later Western blot analysis. The yield of the total amount of protein recovered from each fraction F1, F2, F3, and F4 was between 0-0.006, 0.012-0.024, 0.03-0.06, and 0.18-0.24 mg per heart, respectively.

### Western Blot Analysis

Protein lysates were prepared from frozen hearts homogenized in RIPA lysis buffer. After protein quantification using the BCA protein assay (Bio-Rad Laboratories), lysates were prepared in sample buffer (ThermoFisher) and stored at −20° C. for later analysis. On the date of experimentation, frozen samples were thawed and denatured at room temperature (RT) for 30 min before separation on 4-12% Bis-Tris Gels (NuPAGE, ThermoFisher). After transferring, membranes were fixed in methanol, blocked at RT for 1 hr with 5% BSA in 1×TNT buffer, incubated overnight at 4° C. with primary antibody including rb anti-GAPDH and rb anti-IRAP (Cell Signaling Technology), rb anti-Cav1.2 (Alomone Labs), ms anti-SERCA2a, ms anti-RyR, rb anti-Caveolin 3 (Abcam), ms anti-GLUT4 (Bio-Rad Laboratories), ms anti-Na-K-ATPase (Millipore), and rb anti-BIN13 (Sarcotein Diagnostics). After primary antibody incubation, membranes were washed and incubated with HRP or fluorescent (Alexa 555 or 647) conjugated secondary antibodies (gt anti ms or rb IgG, ThermoFisher) for 1 hr at RT. Immunoreactive bands were imaged with the FluorChem M imager (ProteinSimple) and band intensities were quantified with ImageJ software.

Real-Time Quantitative PCR (qPCR) Analysis

Total RNA was extracted from left ventricular myocardium using PureLink™ RNA extraction kit (Invitrogen), then cDNA was synthesized using SuperScript™ IV VILO™ Master Mix kit (Invitrogen). A custom V5 Ta TaqMan™ probe was designed to detect V5-labeled exogenous cBin1 expression (normalized to housekeeping gene Hprt1) in multiple organs collected from post-treatment mice. A custom-designed porcine cBIN1 TaqMan™ probe was used to detect endogenous cBIN1 in porcine hearts. V5/cBIN1 (porcine) was used to calculate the expression of exogenous cBIN1-V5 as a percentage of endogenous porcine cBIN1. TaqMan™ Universal PCR Master Mix was used for qPCR examination.

### Immunofluorescence Labeling, Spinning Disc Confocal Microscopy, and Imaging Analysis

For myocardial tissue staining, fresh heart cross-sections were embedded in 100% OCT media, flash frozen in dry ice with ethanol, and stored at −80° C. before being sectioned at 10 μm. After acetone fixation, tissue cryosections were permeabilized with 0.1% Triton X-100 (in 5% NGS and 1×PBS) for 1 hr at RT. Freshly isolated live cardiomyocytes were fixed with −20° C. methanol for 5 min and permeabilized with 0.5% Triton X-100 (in 5% NGS and 1×PBS) for 1 hr at RT. For V5, SERCA2a, GLUT4, and IRAP labeling, permeabilized and blocked tissue sections or fixed cardiomyocytes were incubated with primary antibodies against V5 (Sigma-Aldrich), SERCA2a (Abcam), GLUT4 (Bio-Rad Laboratories), or IRAP (Cell Signaling Technology) overnight at 4° C. After washes, fluorescent (Alexa 488, 555, or 647) conjugated secondary antibodies were used to label primary antibody detection before mounting with DAPI containing Prolong® Gold medium. All imaging was obtained with a Nikon Eclipse Ti microscope with a 100×1.49 numerical aperture total internal reflection fluorescence objective and NIS Elements software (Nikon). Confocal Z stacks at Z-step increments of 0.5 μm were collected with a SoRa spinning-disk confocal unit (Yokogawa) connected to the same Ti microscope with diode-pumped solid-state lasers (405, 486, 561, 647) generated from laser merge module 5 (Spectral Applied Research) and captured by a high-resolution SBI digital CMOS camera.

Using the obtained confocal images, SERCA2a, GLUT4, and IRAP fluorescent intensity profiles were generated by ImageJ. For power spectrum analysis, the frequency domain power spectrum of cardiomyocyte image subsections was generated in MatLab using FFT conversion. Normalized t-tubule peak power density (near 1.8-2 μm) was quantified and compared among groups.

### Mass Spectrometry-Based Label Free Quantitative (LFQ) Analysis

LC-MS/MS-based label free quantitation was used to examine global protein abundance from heart lysates obtained from db/m+GFP, db/db+GFP, and db/db+cBIN1 groups (n=4, 4, and 3 hearts for each group, respectively, with 2 technical repeats per heart). Data analysis was performed using MaxQuant (v1.6.7.0) interfaced with the Andromeda search engine and subsequent analysis was done in Perseus (1.6.5.0) to generate 2-tailed t-tests. All proteins were identified by at least one unique peptide present in at least 70% of the replicates for each group with a p value of less than 0.1 by 2-tailed student's t test, and a fold change>1.25 fold between db/db+GFP and db/db+cBIN1 groups were included in the generation of the PCA plot by MetaboAnalyst 5.0. For the analysis of individual proteins, quantitative data for unique peptides were extracted from this dataset for SERCA2a (Atp2a2), RyR₂ (Ryr2), GLUT4 (Slc2a4), IRAP (Lnpep), RAB5a (Rab5a), and RAB10 (Rab10), and then were plotted and compared by 1-way ANOVA followed by Bonferroni's test or Kruskal-Wallis test followed by Dunn's test for comparison between the selected pairs.

Intraperitoneal Glucose and Insulin Tolerance Tests (iGTTs and iITTs)

After fasting for 16 hr for glucose tolerance tests (GTTs) or for 4 hr for insulin tolerance tests (ITTs), basal blood glucose levels were measured via tail bleeding using a glucometer. A minimum of three days separated the glucose and insulin tolerance tests. Mice were administered glucose (1 g/kg, i.p.) or insulin (0.75 IU/kg, i.p.) to assess glucose or insulin tolerance, respectively. Tail blood glucose levels (mg/dL) were subsequently measured at the times indicated.

### Quantification and Statistical Analysis

Data were analyzed using Prism 9 software (GraphPad). All quantitative data were expressed as mean±standard error of the mean (SEM). Normality was assessed by Shapiro-Wilk test. For comparison between two groups, unpaired two-tailed student's t test or nonparametric Mann-Whitney U test was performed. For comparison among three groups, one-way ANOVA followed by Bonferroni's test or nonparametric Kruskal-Wallis test followed by Dunn's test for selected-pair comparison were performed. For assays with multiple insulin concentrations or time points within each group, two-way ANOVA followed by Tukey's or Bonferroni's test for multiple comparisons was used to determine differences among AAV9 groups. Categorical variables were analyzed using Chi-squared tests. P values less than 0.05 were considered statistically significant.

### Example 2

Exogenous cBIN1 Improves Exercise Capacity and Systemic Blood Glucose Control in Diabetic Mice

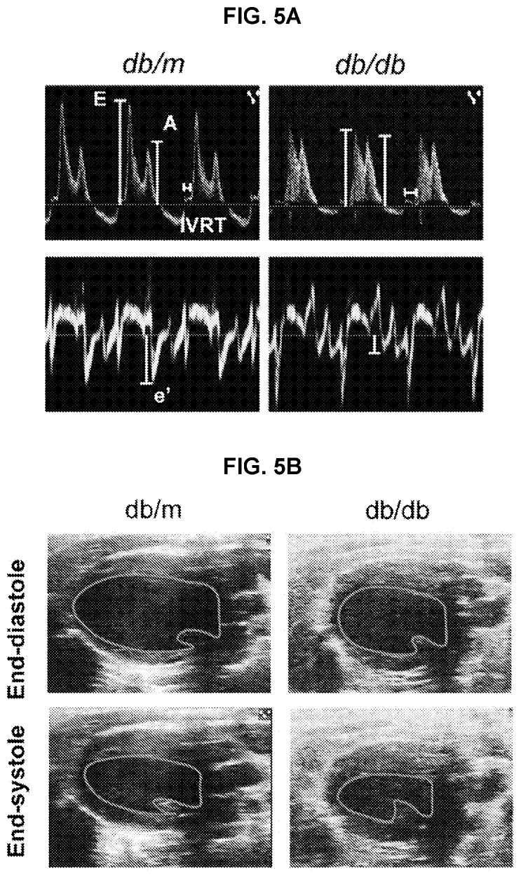

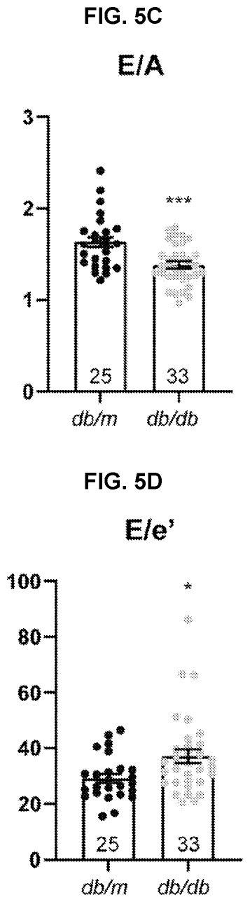

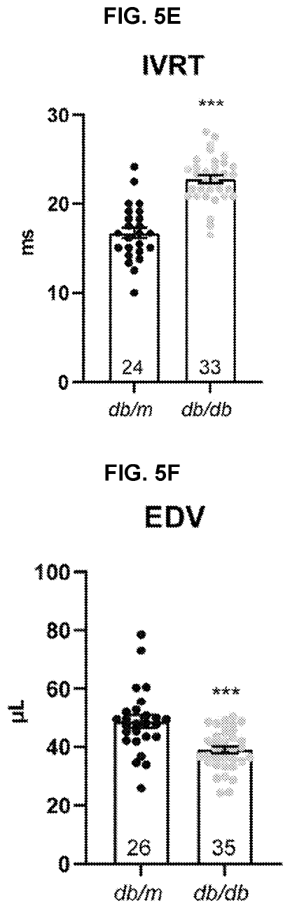

To explore the physiological effect of cBIN1 gene therapy in mice with DCM, leptin receptor-deficient db/db mice were used, which are a common obesity-associated type 2 diabetes (T2D) model. Equal numbers of 9-week-old male and female db/db mice and their lean control littermate db/m mice were injected with V5-tagged mouse cBIN1 (SEQ ID NO: 4) or GFP packaged in AAV9 virus via retro-orbital injection (1×10¹¹ vg in 100 μL of PBS) (n=16-17 per group) (FIG. 1A), studied 8 weeks later for functional assessment, and then euthanized for detailed analysis. Additional groups of db/m and db/db mice (n=5 per group) injected with the same volume (100 μL) of PBS were included as no virus controls (Table 1). The plasma cBIN1 score (CS), which is an index of myocardial remodeling and the inverse of myocardial cBIN1 expression, was increased in 9-week-old db/db mice and was normalized by AAV9-cBIN1 therapy (FIG. 1B). It was found that, unlike control db/m littermates, db/db mice by the age of 9 weeks had already developed phenotypic DCM with impaired exercise tolerance and diastolic dysfunction, as evidenced by alterations in echocardiogram-measured diastolic parameters (i.e., reduced E/A, elevated E/e′, and prolonged isovolumic relaxation time (IVRT)) (FIG. 1C-E, FIG. 5, and Table 2). Administration of AAV9-cBIN1 improved exercise tolerance in db/db mice, as indicated by doubled maximal running distance on a treadmill in cBIN1-treated db/db mice relative to AAV9-GFP treatment (FIG. 1C). Body weight remained increased in db/db mice with no difference observed between GFP and cBIN1 treatment (Table 1), which indicated that improved exercise tolerance in db/db+cBIN1 mice is body weight independent and most likely due to a functional recovery of cardiac hemodynamics. Surprisingly, elevated plasma glucose levels in 17-week-old db/db mice were partially rescued with cBIN1 treatment administered 8 weeks earlier (FIG. 1D). AAV9-cBIN1-improved systemic glycemic control was further validated by improved glucose tolerance (FIG. 1F). On the other hand, plasma insulin levels and insulin tolerance capacity remained similarly impaired between GFP and cBIN1 treatment (FIGS. 1E and 1G).

TABLE 1
Echocardiography parameters in 17-week-old db/m and db/db mice at 8-weeks post-injection of PBS control or AAV9.

| | db/m | | | db/db | | |
|---|---|---|---|---|---|---|
| | PBS | AAV9-GFP | AAV9-cBIN1 | PBS | AAV9-GFP | AAV9-cBIN1 |
| | (n = 5) | (n = 11-12) | (n = 9-10) | (n = 5) | (n = 16) | (n = 16-17) |
| E (mm/s) | 620.49 ± 53.93 | 552.19 ± 31.29 | 577.25 ± 15.85 | 377.02 ± 25.45*** | 413.11 ± 20.74*** | 541.97 ± 13.63^{###, †††} |
| A (mm/s) | 372.36 ± 38.42 | 340.54 ± 30.11 | 359.42 ± 18.92 | 330.34 ± 36.15 | 381.86 ± 22.43 | 324.79 ± 18.00 |
| e′ (mm/s) | 17.05 ± 0.91 | 19.32 ± 1.18 | 18.99 ± 1.38 | 10.04 ± 0.34* | 12.62 ± 0.68*** | 21.39 ± 1.43^{###, †††} |
| E/A | 1.69 ± 0.07 | 1.67 ± 0.07 | 1.63 ± 0.07 | 1.19 ± 0.13** | 1.09 ± 0.03*** | 1.75 ± 0.10^{###, †††} |
| E/e′ | 36.24 ± 1.94 | 30.12 ± 2.97 | 31.75 ± 2.47 | 37.55 ± 2.13 | 33.64 ± 1.95 | 26.59 ± 1.28^{##, ††} |
| MPI | 0.44 ± 0.04 | 0.54 ± 0.03 | 0.51 ± 0.02 | 0.89 ± 0.05*** | 0.78 ± 0.03***^{, #} | 0.48 ± 0.02^{###, †††} |
| IVRT (ms) | 13.78 ± 1.67 | 16.55 ± 1.24 | 17.32 ± 1.06 | 29.86 ± 1.32*** | 24.50 ± 0.90***^{, ##} | 15.22 ± 0.53^{###, †††} |
| EF (%) | 51.71 ± 2.72 | 52.79 ± 3.11 | 60.91 ± 2.98*^{, †} | 64.30 ± 2.15* | 62.16 ± 1.74** | 66.67 ± 1.43 |
| LVEDV (μL) | 68.91 ± 2.88 | 61.85 ± 4.38 | 53.56 ± 4.17^{#} | 39.28 ± 1.43*** | 36.77 ± 1.53*** | 52.27 ± 2.80^{#, †††} |
| LVESV (μL) | 33.49 ± 2.81 | 29.75 ± 3.34 | 21.77 ± 3.20^{##, †} | 14.13 ± 1.30*** | 14.14 ± 1.05*** | 17.84 ± 1.49 |
| HR (bpm) | 397.25 ± 17.94 | 438.26 ± 7.48 | 424.10 ± 19.88 | 410.16 ± 13.12 | 443.54 ± 9.57 | 444.31 ± 11.37 |
| SV (μL) | 35.42 ± 1.41 | 32.10 ± 2.57 | 31.79 ± 1.47 | 25.15 ± 0.53** | 22.63 ± 0.80*** | 34.43 ± 1.51^{##, †††} |
| CO (mL/min) | 14.11 ± 0.95 | 15.03 ± 1.82 | 13.50 ± 0.93 | 10.32 ± 0.37 | 10.00 ± 0.34** | 15.06 ± 0.68^{††} |
| LVAWs (mm) | 1.37 ± 0.07 | 1.22 ± 0.05 | 1.21 ± 0.03 | 1.77 ± 0.06*** | 1.36 ± 0.06*^{, ###} | 1.46 ± 0.04**^{, ##} |
| LVAWd (mm) | 0.88 ± 0.09 | 0.79 ± 0.05 | 0.81 ± 0.03 | 0.96 ± 0.05 | 0.90 ± 0.04* | 0.87 ± 0.02 |
| LVPWs (mm) | 1.05 ± 0.06 | 1.03 ± 0.05 | 1.08 ± 0.07 | 1.56 ± 0.13*** | 1.25 ± 0.05**^{, ##} | 1.32 ± 0.05**^{, #} |
| LVPWd (mm) | 0.78 ± 0.03 | 0.76 ± 0.05 | 0.76 ± 0.06 | 0.95 ± 0.07 | 0.84 ± 0.03 | 0.87 ± 0.04 |
| RWT | 0.35 ± 0.02 | 0.37 ± 0.02 | 0.38 ± 0.03 | 0.59 ± 0.06*** | 0.47 ± 0.01**^{, ##} | 0.46 ± 0.02*^{, ##} |
| LVM (mm) | 147.38 ± 8.65 | 120.87 ± 8.78^{#} | 117.17 ± 8.58^{#} | 110.44 ± 5.47* | 112.58 ± 6.09 | 122.63 ± 5.51 |
| BW (g) | 28.30 ± 0.65 | 27.25 ± 1.06 | 26.89 ± 0.62 | 50.12 ± 2.20*** | 52.50 ± 0.87*** | 52.85 ± 0.87*** |

E, peak velocity blood flow from left ventricular relaxation in early diastole; A, peak velocity flow in late diastole caused by atrial contraction; e′, early diastolic mitral annular tissue velocity; MPI, myocardial performance index; IVRT, isovolumic relaxation time; EF, ejection fraction; LVEDV, left ventricular end-diastolic volume; LVESV, left ventricular end-systolic volume; HR, heart rate; SV, stroke volume; CO, cardiac output; LVAWs/LVPWs, left ventricular end-systolic anterior/posterior wall thickness; LVAWd/LVPWd, left ventricular end-diastolic anterior/posterior wall thickness; RWT, relative wall thickness; LVM, left ventricular mass; BW, body weight.
Data are presented as mean ± SEM. Two-way ANOVA followed by Fisher LSD test was used for comparison among groups.
*, **, ***indicates p < 0.05, p < 0.01, or p < 0.001 when compared between two different genotype groups within the same AAV9 treatment.
^{#}, ^{##}, ^{###}indicates p < 0.05, p < 0.01, or p < 0.001 when compared to PBS group within the same genotype.
^{†}, ^{††}, ^{†††}indicates p < 0.05, p < 0.01, or p < 0.001 when compared to AAV9-GFP treatment group within the same genotype.

TABLE 2
Echocardiography parameters in 9-week-old db/m and db/db mice.

| | db/m | db/db |
|---|---|---|
| | (n = 24-26) | (n = 33-35) |
| E (mm/s) | 496.8 ± 21.26 | 465.9 ± 9.98 |
| A (mm/s) | 316.6 ± 18.14 | 346.6 ± 12.28 |
| e′ (mm/s) | 18.12 ± 1.13 | 13.72 ± 0.63*** |
| E/A | 1.63 ± 0.06 | 1.38 ± 0.04*** |
| E/e′ | 29.22 ± 1.60 | 37.08 ± 2.46* |
| MPI | 0.51 ± 0.02 | 0.66 ± 0.01*** |
| IVRT (ms) | 16.73 ± 0.65 | 22.77 ± 0.46*** |
| EF (%) | 54.93 ± 1.59 | 58.12 ± 1.81 |
| LVEDV (μL) | 48.64 ± 2.18 | 39.02 ± 1.18*** |
| LVESV (μL) | 22.02 ± 1.29 | 16.66 ± 1.04** |
| HR (bpm) | 412.9 ± 7.40 | 420.7 ± 7.35 |
| SV (μL) | 26.62 ± 1.38 | 22.35 ± 0.71* |
| CO (mL/min) | 10.99 ± 0.59 | 9.41 ± 0.34* |
| LVAWs (mm) | 1.12 ± 0.04 | 1.28 ± 0.03** |
| LVAWd (mm) | 0.74 ± 0.03 | 0.79 ± 0.02 |
| LVPWs (mm) | 0.95 ± 0.04 | 1.03 ± 0.02 |
| LVPWd (mm) | 0.70 ± 0.02 | 0.71 ± 0.01 |
| RWT | 0.36 ± 0.01 | 0.38 ± 0.01 |
| LVM (mm) | 97.46 ± 4.69 | 98.01 ± 2.23 |
| BW (g) | 24.08 ± 0.84 | 43.53 ± 0.75*** |

E, peak velocity blood flow from left ventricular relaxation in early diastole; A, peak velocity flow in late diastole caused by atrial contraction; e′, early diastolic mitral annular tissue velocity; MPI, myocardial performance index; IVRT, isovolumic relaxation time; EF, ejection fraction; LVEDV, left ventricular end-diastolic volume; LVESV, left ventricular end-systolic volume; HR, heart rate; SV, stroke volume; CO, cardiac output; LVAWs/LVPWs, left ventricular end-systolic anterior/posterior wall thickness; LVAWd/LVPWd, left ventricular end-diastolic anterior/posterior wall thickness; RWT, relative wall thickness; LVM, left ventricular mass; BW, body weight.
Data are presented as mean ± SEM. Unpaired Student's t-test was used for comparison between db/m and db/db.
*, **, ***indicates p < 0.05, p < 0.01, or p < 0.001 when compared between the two groups.

The CMV-promoted AAV9 vector is well-established for cardiac tropism with retained cardiac selectivity at a relatively low dose of 1×10¹¹ vg, as was used in this study. To further confirm cardiac-specific gene transfer by AAV9-cBIN1, a Taqman probe was developed for detecting the V5 sequence included only in the exogenous gene transduced by AAV9. As illustrated in FIG. 1H, quantitative real-time PCR analysis of cDNA samples obtained across nine different organs (heart, liver, skeletal muscle, pancreas, kidney, spleen, lung, and adipose) identified that exogenous cBIN1-V5 (SEQ ID NO: 4) was only effectively transduced in hearts, demonstrating selectivity. These data indicated that improved exercise capacity and systemic glycemic control in db/db mice originates from cardiac-limited transduction of exogenous cBIN1. Cardiomyocyte expression of cBIN1 was further examined at the protein level by western blotting and immunofluorescence analysis. Consistent with plasma CS results (FIG. 1B), western blotting analysis of myocardial tissue lysates identified that cardiac cBIN1 expression was reduced in db/db+GFP hearts and was normalized by AAV9-cBIN1 treatment (FIG. 1I). Immunofluorescent labeling with anti-V5 antibody followed by spinning-disc confocal imaging detected transduction of exogenous cBIN1-V5 protein in nearly 100% of cardiomyocytes in db/db mouse hearts (FIG. 6).

### Example 3

Exogenous cBIN1 Normalizes Membrane Microdomains at t-Tubules

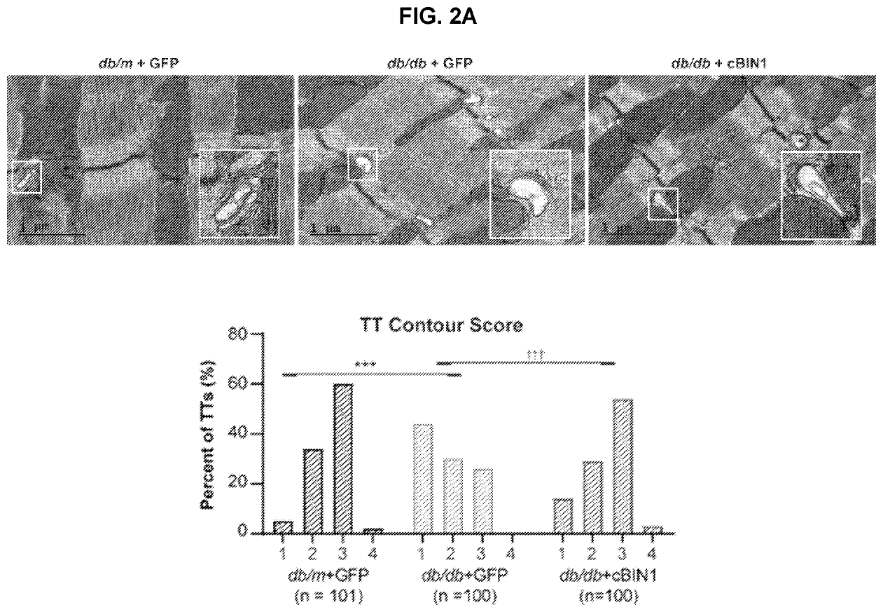

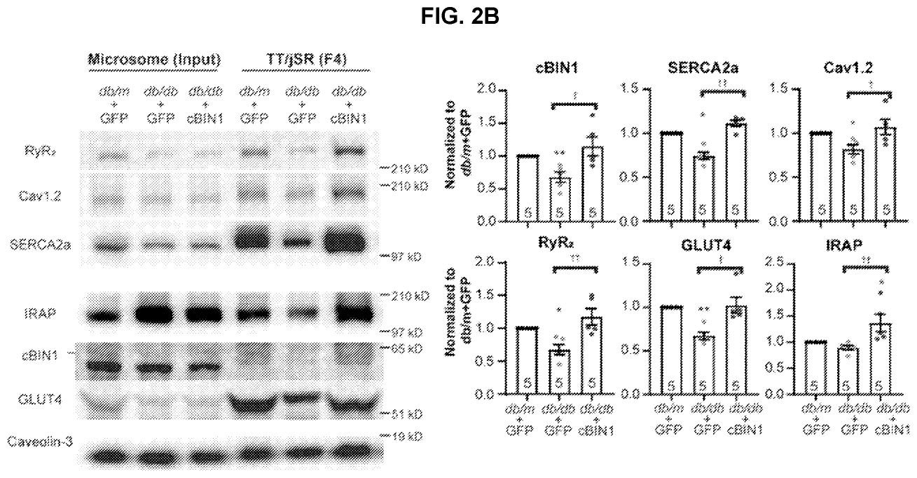

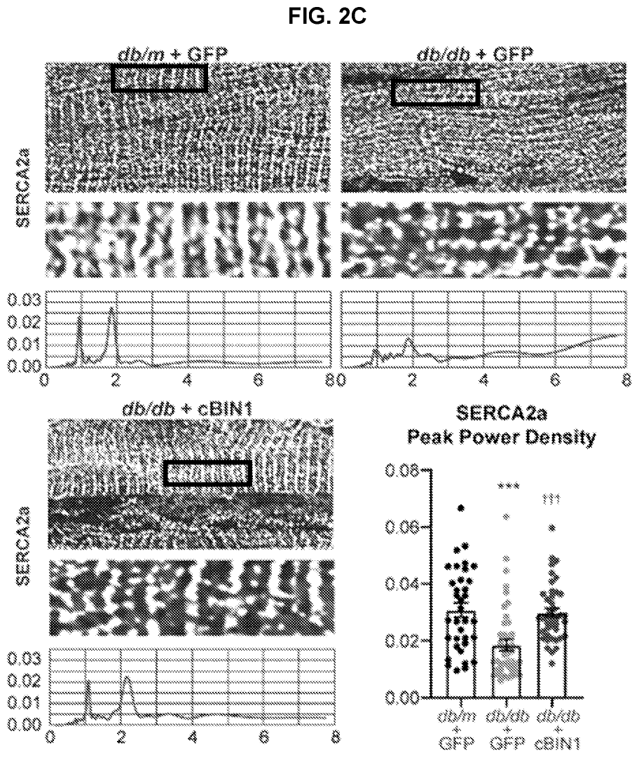

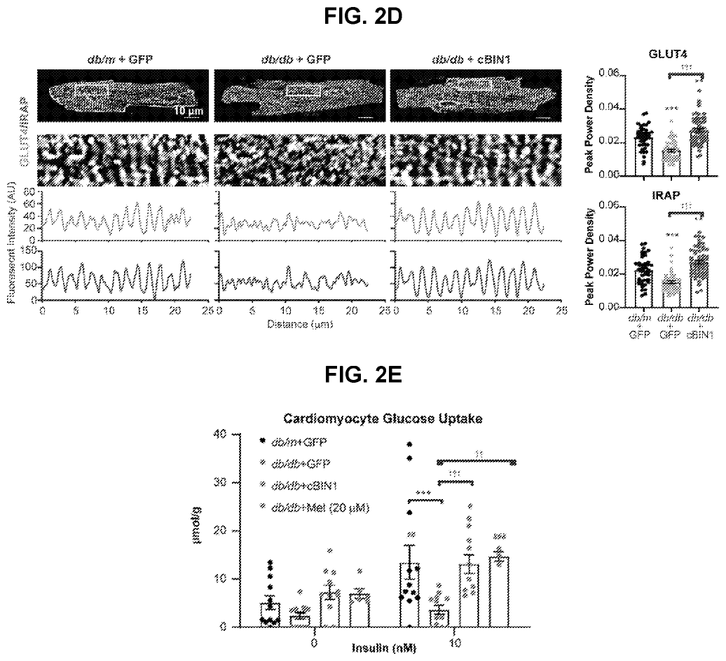

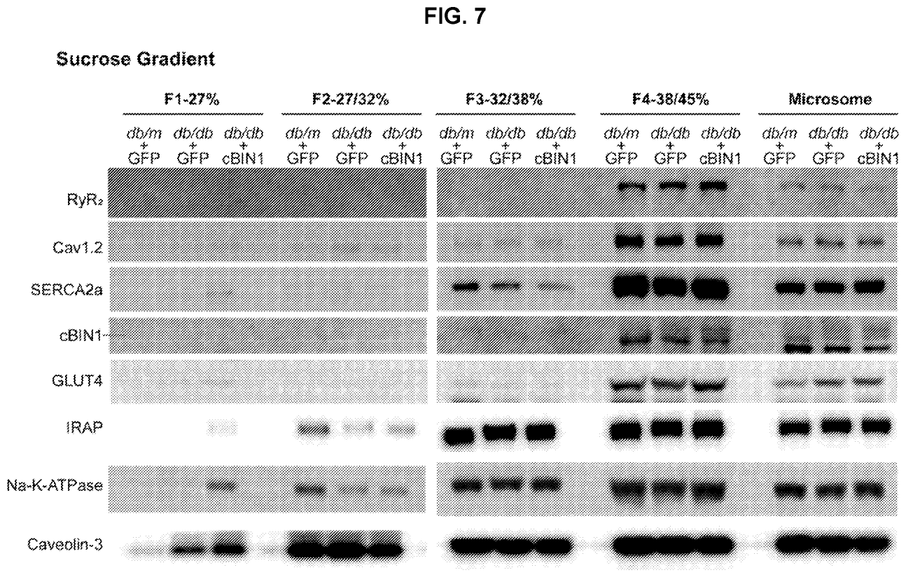

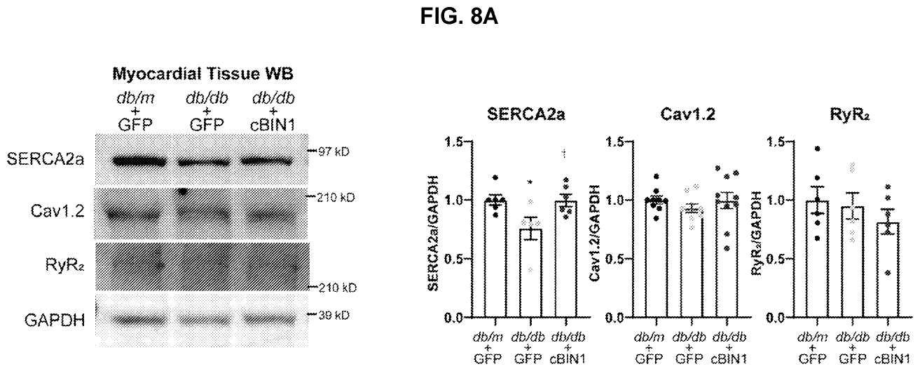

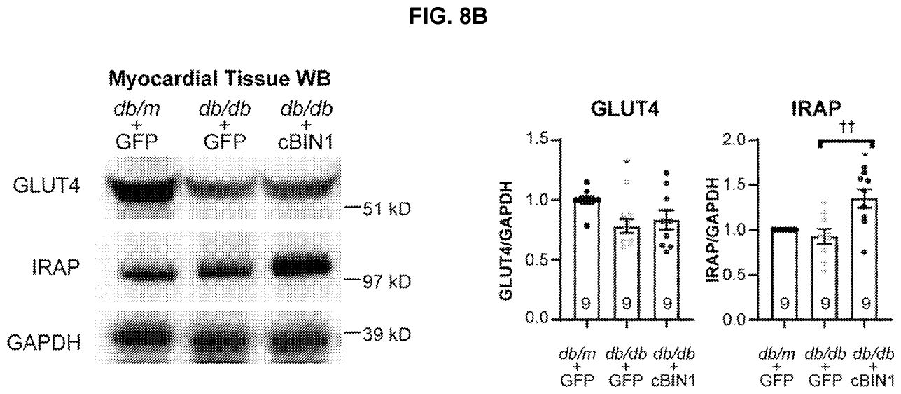

Given the capacity of cBIN1 to form t-tubule membrane microfolds, and that cBIN1 levels decreased during DCM progression (FIG. 1), t-tubule membrane ultrastructure of diabetic cardiomyocytes was explored in response to cBIN1 gene therapy. Using transmission electron microscopy (TEM) imaging, it was found that decreased cBIN1 in db/db cardiomyocytes was associated with loss of t-tubule microfolds, as quantified by a t-tubule contour score, which was restored by cBIN1 gene therapy (FIG. 2A). Next, biochemical sucrose gradient-based fractionation of cardiac microsomes was used to evaluate subcellular distribution of proteins associated with microsomes originated from cBIN1-microfolds. The 38%/45% sucrose gradient interface (fraction 4, F4) had the highest cBIN1 yield, indicating F4 as the fraction originating from cBIN1-microfolds. The same cBIN1-enriched microsome F4 fraction was also found to contain TT/jSR proteins including Cav1.2 (the pore forming subunit of L-type calcium channel, LTCC), ryanodine receptor 2 (RyR₂), and sarcoplasmic reticulum calcium-ATPase 2a (SERCA2a) (FIG. 7). These data were consistent with previous results that cBIN1-microfolds house both the systolic calcium releasing unit formed by LTCC-RyR₂ dyads, and a subpopulation of SERCA2a. In cardiac microsomes prepared from db/db hearts with reduced cBIN1, there was a significant reduction in the amount of the F4-located jSR subpopulation of SERCA2a (FIG. 2B). Total SERCA2a expression was also decreased, which could be restored with cBIN1 treatment (FIG. 8A). Immunofluorescent imaging with power spectrum analysis further confirmed that intracellular organization of SERCA2a became disorganized in db/db mouse cardiomyocytes and was normalized by AAV9-cBIN1 gene therapy (FIG. 2C).

Furthermore, in db/db hearts with preserved systolic function, there were no significant changes in the overall myocardial expression of LTCCs and RyRs (FIG. 8A), while the concentration of LTCCs and RyRs in the F4-TT/jSR fraction was readily increased in response to exogenous cBIN1 treatment (FIG. 2B). These data indicated that targeting cBIN1 microdomains in diabetic cardiomyocytes, similar to sympathetic-overdriven cardiomyocytes with diastolic dysfunction, can organize calcium handling machinery (e.g., SERCA2a, Cav1.2, RyR₂), effective for the total concentration of and, to a greater extent, the localization of critical proteins to t-tubule membranes.

In addition to calcium handling proteins, a significant concentration of glucose transporter 4 (GLUT4) protein to the same F4-TT/jSR fraction was enriched with cBIN1-microsomes (FIG. 2B). T-tubule is a common membrane target of insulin and contraction-stimulated translocation of the GLUT4 protein in cardiac muscle. Thus, in addition to serving as trafficking hubs for calcium handling machinery, cBIN1-microfolds at t-tubules may also provide ultrastructural foundations to attract GLUT4 surface translocation upon insulin stimulation. Basally, GLUT4 is present mainly in intracellular GLUT4 storage vesicles (GSVs). Upon insulin stimulation or muscle contraction, GLUT4 is translocated to the surface membrane to enable a rapid increase in glucose uptake. Impaired GLUT4 expression and translocation in insulin-sensitive tissues contributes to the hyperglycemia observed in diabetes.

Alterations of GLUT4 expression and localization were explored in db/db cardiomyocytes in response to AAV9-cBIN1 treatment. While AAV9-cBIN1 did not alter total GLUT4 protein expression, which was slightly reduced in db/db hearts (FIG. 8B), it significantly increased GLUT4 localization to the t-tubule membrane, as indicated by both biochemical fractionation (FIG. 2B) and fluorescent imaging analysis of isolated cardiomyocytes (FIG. 2D). Given that insulin-responsive aminopeptidase (IRAP) participates in GSV trafficking in insulin sensitive tissues, the protein expression and subcellular distribution of IRAP were examined as well. Exogenous cBIN1 increased IRAP expression in whole heart lysates and the TT/jSR region, as indicated by membrane fractionation and fluorescent confocal imaging results (FIG. 8B, and FIGS. 2B and 2D). The functional consequence of altered GLUT4 membrane localization should be altered glucose uptake, which can be measured by insulin-dependent cardiomyocyte glucose uptake assays. Cardiomyocytes were isolated and, after starvation, insulin-stimulated uptake of 2-Deoxy-D-glucose was measured. The db/db+GFP cardiomyocytes had a profound loss of glucose uptake in the presence of 10 nM insulin, but uptake was normalized by AAV9-cBIN1 pre-treatment. In fact, cBIN1 rescue of glucose uptake was quantitatively comparable to the rescue induced by the glucose-lowering drug, metformin (20 M), which is known to increase cell surface expression of GLUT4 in cardiomyocytes (FIG. 2E).

The GLUT4 trafficking data (FIG. 2D) together with improved blood glucose tolerance (FIG. 1F) with reduced hyperglycemia (FIG. 1D) following cBIN1 treatment in db/db mice, and the ability of cardiomyocytes to take up glucose (FIG. 2E), collectively point to a possibility of direct cardiomyocyte contribution to blood glucose regulation in patients with T2DM. To test the relationship between cBIN1 and GLUT4 expression without the complication of T2DM, glucose uptake in mice was analyzed with cardiac-specific heterozygous Bin1 deletion (Bin1 HT, Bin 1^{fl/+}; Myh6-Cre⁺). Bin1 HT mice have depleted t-tubule microfolds in cardiomyocytes and are sensitive to pressure overload. When Bin1 HT mice are more than 1 year old, they develop a significant reduction in E/A (1.04±0.05 versus WT 1.58±0.15, P<0.01) that can be rescued by AAV9-cBIN1 (1.72±0.07, P<0.01 when compared with Bin1 HT).

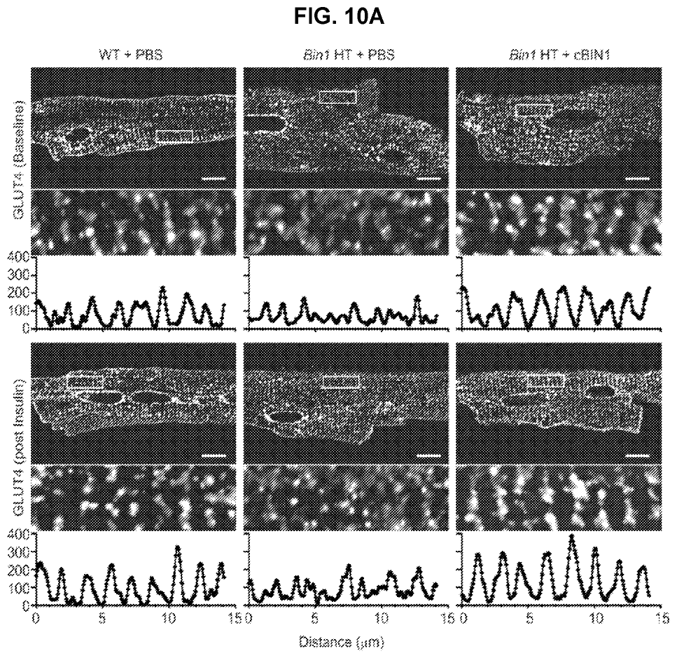

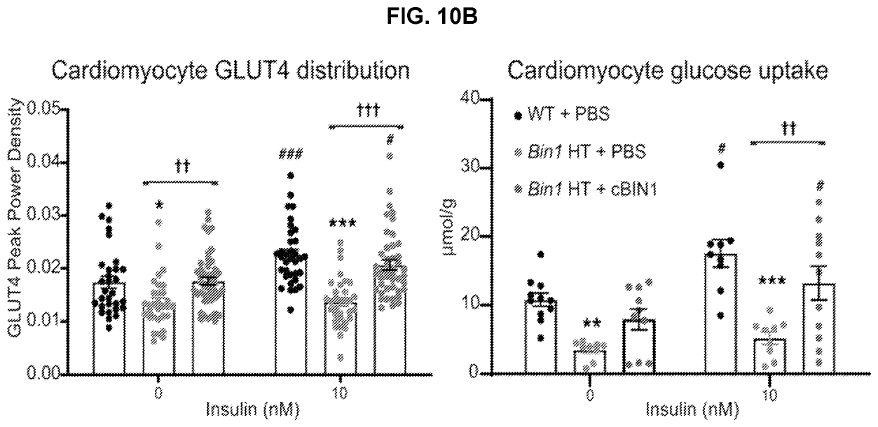

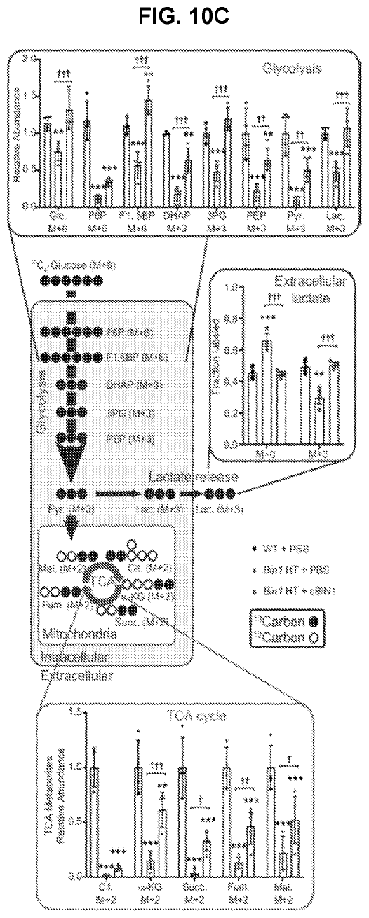

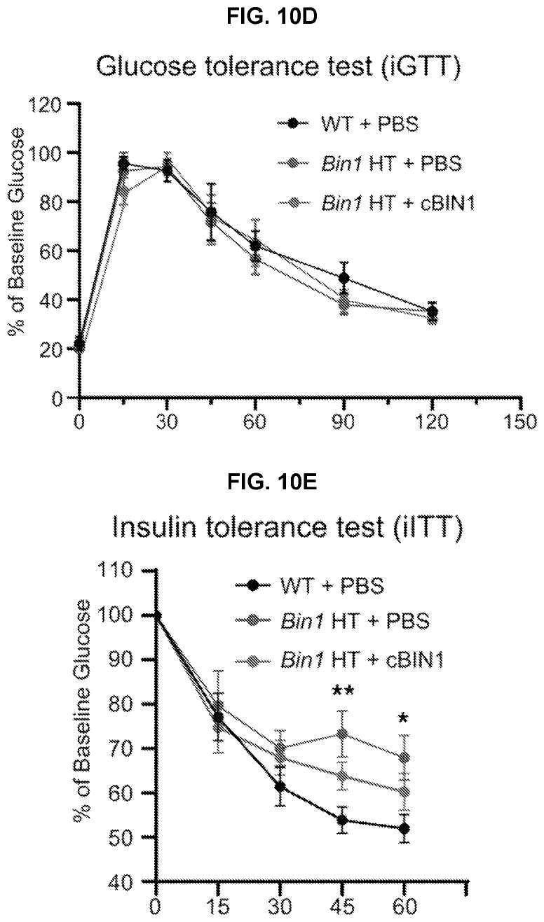

Intracellular distribution of GLUT4 and 2DG uptake were explored in Bin1 HT cardiomyocytes (with or without prior in vivo AAV9-cBIN1 rescue) following 10 nM insulin stimulation for 20 minutes. Consistent with results from db/db mice, Bin1 HT cardiomyocytes lost organized intracellular GLUT4 distribution to the TT/jSR region (FIG. 10A-B) with a functional consequence of reduced 2DG uptake (FIG. 10B), which normalized with AAV9-cBIN1 therapy. With reduced glucose uptake, Bin1 HT cardiomyocytes likely have impaired glucose oxidation, providing fuel for the mitochondrial tricarboxylic acid (TCA) cycle (schematic in FIG. 10C). Therefore, the glucose utilization was tracked using ¹³C₆-glucose tracing in isolated Bin1 HT cardiomyocytes. Consistent with impaired glucose uptake, there was an overall loss of ¹³C-enrichment in intracellular glycolytic and TCA cycle intermediates from labeled glucose in Bin1 HT cardiomyocytes (FIG. 10C). Furthermore, a resultant decreased release of heavy-labeled lactate (M+3) from Bin1 HT cardiomyocytes into the extracellular bathing medium was also observed (FIG. 10C). With prior in vivo AAV9-cBIN1 treatment, the abnormalities in glucose tracing could be rescued. Moreover, in these Bin1 HT lean mice with still-preserved glucose tolerance (FIG. 10D), in vivo insulin administration resulted in less reduction in blood glucose levels, which could be improved with prior in vivo rescue with AAV9-cBIN1 (FIG. 10E). These data, together with results from db/db mice, indicate that TT/jSR localized cBIN1 microdomains may serve as a trafficking destination of GSVs, contributing to systemic glycemic control.

### Example 4

### AAV9-cBIN1 Downregulates Myocardial Mitochondrial Components and Improves Mitochondrial Function in Diabetic Cardiomyocytes

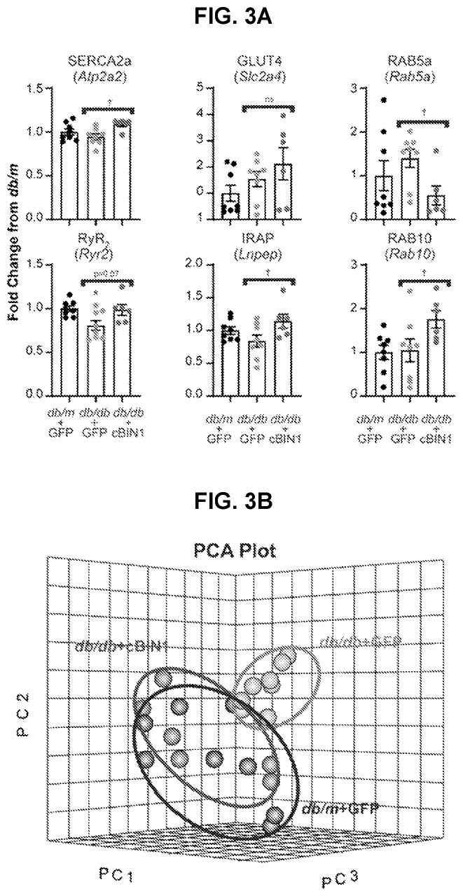

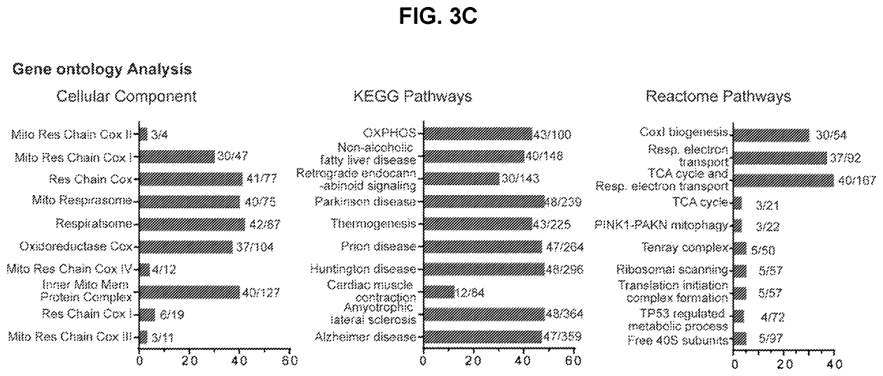

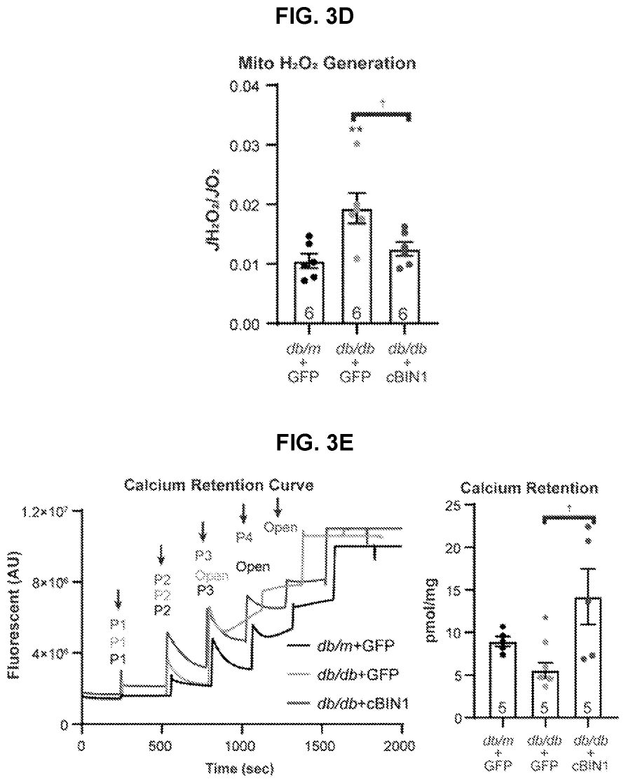

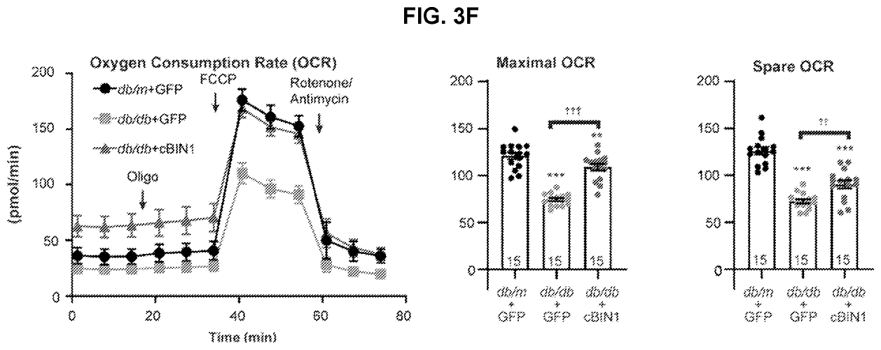

cBIN1 microdomains at t-tubules may serve as a centralized trafficking and signaling hub for coordinated beat-to-beat cytosolic calcium handling and glucose uptake and metabolism in cardiomyocytes. To generate a proteomic profile of diabetic myocardium in response to cBIN1 gene therapy, LC-MS/MS-based analysis of cardiac tissue from 8 weeks post-injection mice was performed. From these data, individual proteins were targeted for calcium handling proteins (SERCA2a and RyR₂) and GSV proteins (GLUT4 and IRAP). Consistent with western blotting results, there was a significant increase in myocardial expression of SERCA2a (1.2-fold) and IRAP (1.4-fold) with cBIN1 gene therapy when compared with control GFP treated db/db hearts (FIG. 3A). Interestingly, cBIN1 gene therapy induced a 1.7-fold increase of RAB10, which is required for insulin-induced GSV translocation to the plasma membrane, as well as a 2.5-fold decrease of RAB5a, which is responsible for endocytosis and surface removal of GLUT4 from the plasma membrane. These data provided additional proteomics evidence for the ability of cBIN1 gene therapy to restore intracellular calcium handling and insulin-dependent glucose uptake in cardiomyocytes (FIG. 2).

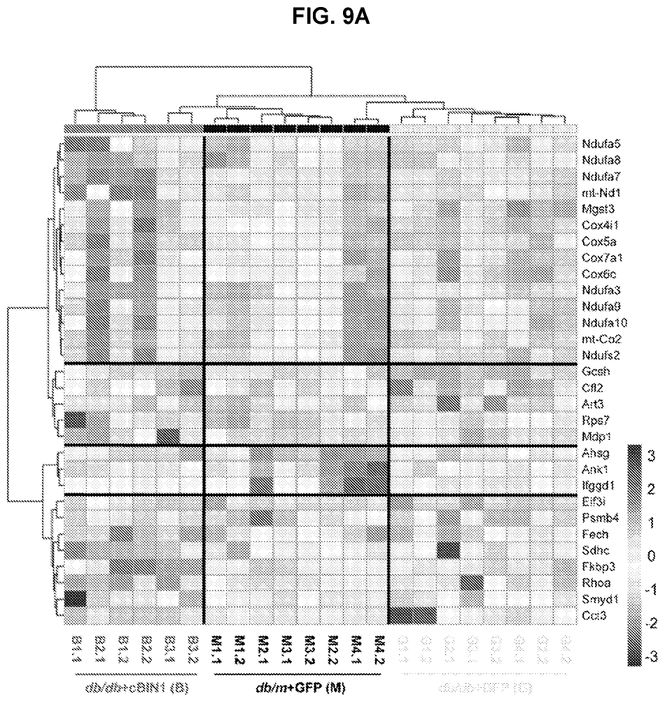

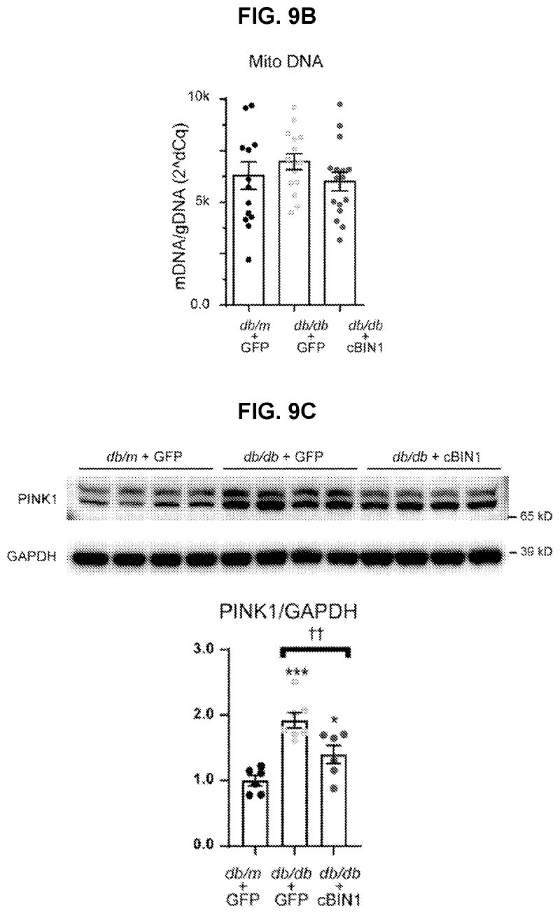

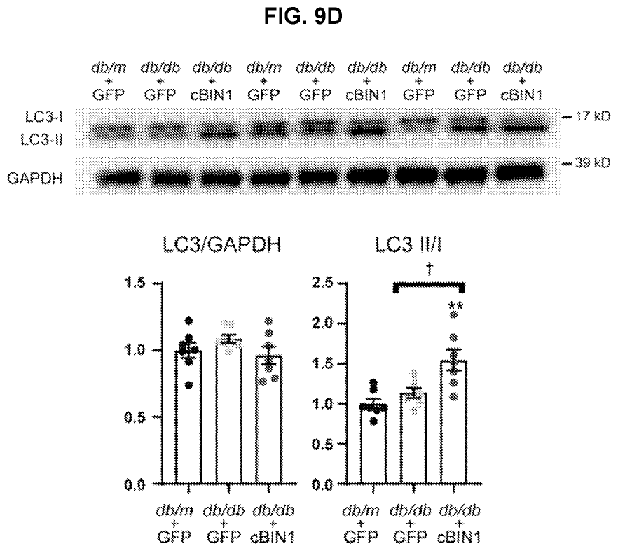

Additionally, as indicated in the proteomic principal component analysis (PCA) plot (FIG. 3B), the overall protein segregation of db/db+GFP hearts from db/m+GFP hearts was nearly normalized by cBIN1 gene therapy. A heatmap generated with the top 30 most significantly changed proteins in db/db hearts with cBIN1 treatment revealed that most affected proteins were associated with mitochondria (FIG. 9A). LFQ results indicated that oxidative phosphorylation (OXPHOS) complexes, particularly complex I components (i.e., Ndufa7, mt-Nd1, Ndufa9, etc.), were increased in db/db hearts and normalized with cBIN1 treatment. STRING database analysis of the 79 genes with a significant difference between GFP and cBIN1 treatment in db/db hearts (p<0.05, Fold change>=1.5) also identified that the 10 most enriched gene ontology terms for cellular components included mitochondrial respiratory chain complexes, with the top KEGG pathway as the OXPHOS pathway, and the top reactome pathways of mitochondrial respiration and metabolism (FIG. 3C). Mitochondrial DNA was not increased in db/db hearts (FIG. 9B); however, western blotting analysis identified a significant elevation of PINK1 in db/db hearts (FIG. 9C), suggesting mitochondrial damage in diabetic hearts. cBIN1 treatment significantly activated LC3-II/I with decreased PINK1 in db/db hearts (FIG. 9C-D) and preserved mitochondrial DNA (FIG. 9B), indicating elimination of damaged mitochondria and a resultant healthier pool of cardiac mitochondria after receiving cBIN1 gene therapy.

Reactive oxygen species (ROS) production and calcium retention capacity were examined in mitochondria isolated from db/db hearts. Mitochondrial calcium retention capacity describes the maximal calcium uptake by mitochondria before the opening of permeability transition pores, which is commonly used for evaluating mitochondrial damage under stress. In diabetic db/db hearts, damaged mitochondria generated increased H₂O₂ with reduced calcium retention capacity, indicating impaired mitochondrial function with worse oxidative stress (FIG. 3D-E). AAV9-cBIN1 treatment was sufficient to reduce mitochondrial H₂O₂ production with improved calcium retention in mitochondria. At the level of whole cardiomyocytes, seahorse-measured maximal and spare oxygen consumption rates of db/db mouse cardiomyocytes were also significantly reduced when compared to those of non-diabetic db/m cardiomyocytes (FIG. 3F), with rescue by cBIN1 gene therapy. These data indicated that AAV9-cBIN1 gene therapy can normalize mitochondrial stress and cellular respiration in db/db cardiomyocytes, protecting mitochondrial function and limiting oxidative stress in diabetic cardiomyocytes.

### Example 5

Exogenous cBIN1 Rescues Diastolic Heart Failure in Diabetic Mice

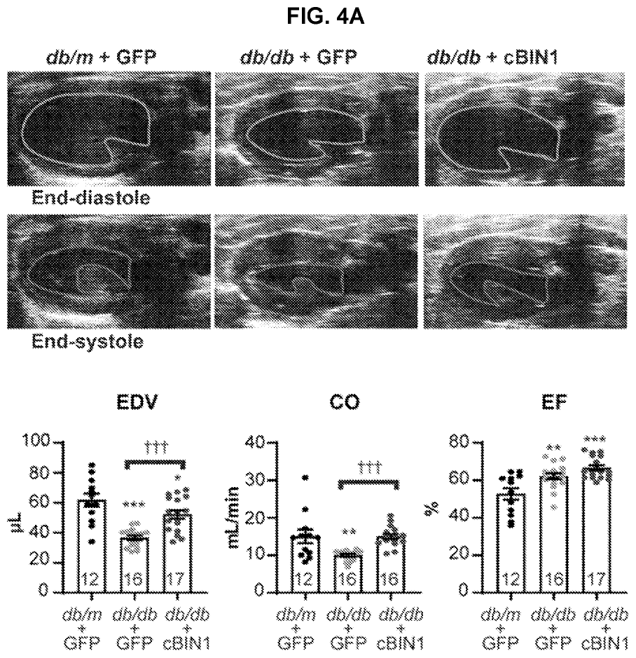

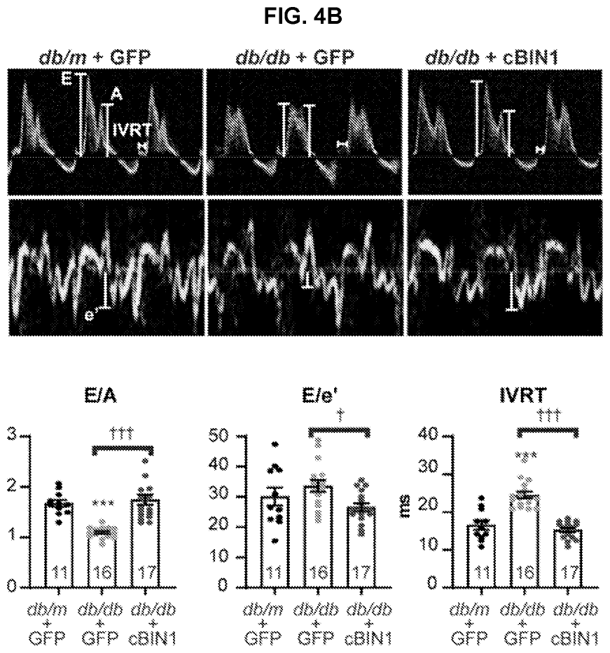

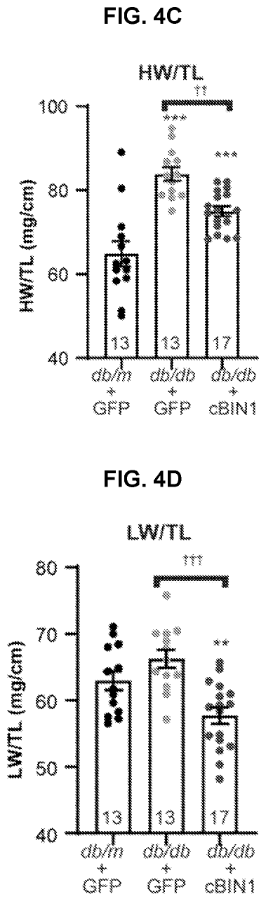

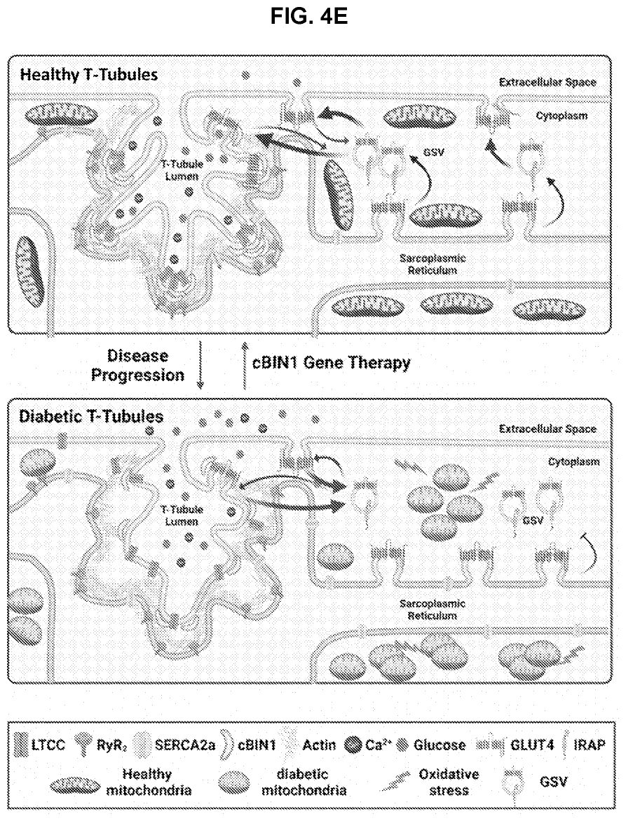

It was explored whether the cBIN1-induced rescue of intracellular calcium handling, glucose uptake, and mitochondrial stress of diabetic cardiomyocytes could translate to in vivo cardiac functional performance in cBIN1-treated db/db mice. Post-treatment echocardiograms obtained from 17-week-old mice with AAV9 administered 8 weeks earlier were evaluated for cardiac geometry, wall thickness, as well as left ventricular contractile and diastolic function. As indicated in the representative two-dimensional long-axis view images of the left ventricles (FIG. 4A), db/db mouse hearts had an impaired diastolic phenotype with a significant concentric hypertrophy, as evidenced by increased wall thickness and reduced ventricular volume (Table 1), yet with a preserved ejection fraction. With AAV9-cBIN1 treatment in db/db mice, reduced left ventricular end diastolic volume (EDV), stroke volume (SV), and cardiac output (CO) were all normalized (Table 1 and FIG. 4A). For diastolic functional analysis, FIG. 4B contains representative pulse-wave Doppler images of E and A waves (top panel images) and tissue Doppler images of e′ velocity (bottom panel images). The summarized E/A, E/e′, and IVRT are included in the bar graphs at the bottom of FIG. 4B. When compared to db/m mice, db/db mice displayed a significant decrease in the E/A ratio, prolongation of IVRT, and impairment of myocardial performance index (MPI) (Table 1 and FIG. 4B). AAV9-cBIN1 treatment also rescued E/A and MPI, shortened IVRT, and significantly reduced E/e′ in db/db mice. Post-mortem analysis identified that the elevated heart and lung weight (expressed as a ratio to tibial length, HW/TL, LW/TL) of db/db mice were both reduced with cBIN1 treatment (FIG. 4C-D), further indicating that cBIN1 gene therapy improved diabetic cardiomyopathy-associated cardiac hypertrophy and lung edema. Moreover, in 8-month-old db/db mice with worsening diabetic cardiomyopathy, it was further confirmed that AAV9-cBIN1 gene therapy improved cardiac function and exercise intolerance (Table 3).

TABLE 3
Echocardiography parameters and treadmill performance in 8-months old
db/m and db/db mice at 8-weeks post AAV9-CMV-GFP/cBIN1 injection.

| | db/m-GFP | db/db-GFP | db/db-cBIN1 |
|---|---|---|---|
| | (n = 8) | (n = 7) | (n = 8) |
| E (mm/s) | 576.0 ± 49.09 | 504.8 ± 44.30 | 536.2 ± 38.44 |
| A (mm/s) | 351.3 ± 32.92 | 356.5 ± 20.37 | 344.8 ± 19.18 |
| e′ (mm/s) | 18.32 ± 1.94 | 14.81 ± 2.06 | 20.97 ± 1.07^{†} |
| E/A | 1.67 ± 0.09 | 1.44 ± 0.14 | 1.57 ± 0.11 |
| E/e′ | 33.11 ± 3.76 | 36.93 ± 4.74 | 25.80 ± 1.82^{†} |
| MPI | 0.55 ± 0.03 | 0.71 ± 0.03* | 0.56 ± 0.04^{†} |
| IVRT (ms) | 21.72 ± 0.81 | 28.15 ± 1.02*** | 21.48 ± 0.62^{†††} |
| EF (%) | 70.93 ± 3.49 | 74.20 ± 2.04 | 75.63 ± 1.68 |
| LVEDV (μL) | 50.10 ± 3.29 | 38.86 ± 2.40* | 46.77 ± 3.25 |
| LVESV (μL) | 14.46 ± 1.81 | 10.25 ± 1.28 | 11.27 ± 1.46 |
| HR (bpm) | 437.0 ± 17.61 | 425.4 ± 26.76 | 420.3 ± 10.37 |
| SV (μL) | 35.65 ± 2.65 | 28.61 ± 1.27* | 35.51 ± 2.27† |
| CO (mL/min) | 15.44 ± 1.02 | 12.18 ± 0.97* | 14.80 ± 1.13 |
| LVAWs (mm) | 1.81 ± 0.06 | 1.88 ± 0.05 | 1.78 ± 0.04 |
| LVAWd (mm) | 1.18 ± 0.06 | 1.11 ± 0.07 | 1.07 ± 0.04 |
| LVPWs (mm) | 1.35 ± 0.05 | 1.32 ± 0.05 | 1.34 ± 0.09 |
| LVPWd (mm) | 0.78 ± 0.03 | 0.80 ± 0.04 | 0.78 ± 0.03 |
| RWT | 0.48 ± 0.03 | 0.45 ± 0.03 | 0.45 ± 0.02 |
| LVM (mm) | 118.8 ± 11.32 | 126.4 ± 5.72 | 114.8 ± 4.38 |
| BW (g) | 29.66 ± 0.86 | 40.94 ± 1.70*** | 40.76 ± 2.17*** |
| Maximal Distance (m) | 191.1 ± 3.92 | 25.49 ± 4.56*** | 49.03 ± 7.24***^{, ††} |

E, peak velocity blood flow from left ventricular relaxation in early diastole; A, peak velocity flow in late diastole caused by atrial contraction; e′, early diastolic mitral annular tissue velocity; MPI, myocardial performance index; IVRT, isovolumic relaxation time; EF, ejection fraction; LVEDV, left ventricular end-diastolic volume; LVESV, left ventricular end-systolic volume; HR, heart rate; SV, stroke volume; CO, cardiac output; LVAWs, left ventricular end-systolic anterior wall thickness; LVAWd, left ventricular end-diastolic anterior wall thickness; LVPWs, left ventricular end-systolic posterior wall thickness; LVPWd, left ventricular end-diastolic posterior wall thickness; RWT, relative wall thickness; LVM, left ventricular mass; BW, body weight; HW/TL, heart weight/ tibial length; and LW/TL, lung weight/tibial length.
Data are presented as mean ± SEM. One-way ANOVA followed by Bonferroni's test or Kruskal-Wallis test followed by Dunn's test was performed for selected pair comparison.
*, **, ***p < 0.05, 0.01, or 0.001 for comparison vs. db/m-GFP; ^{†}, ^{††}, ^{†††}p < 0.05, 0.01, or 0.001 for comparison vs. db/db-GFP.

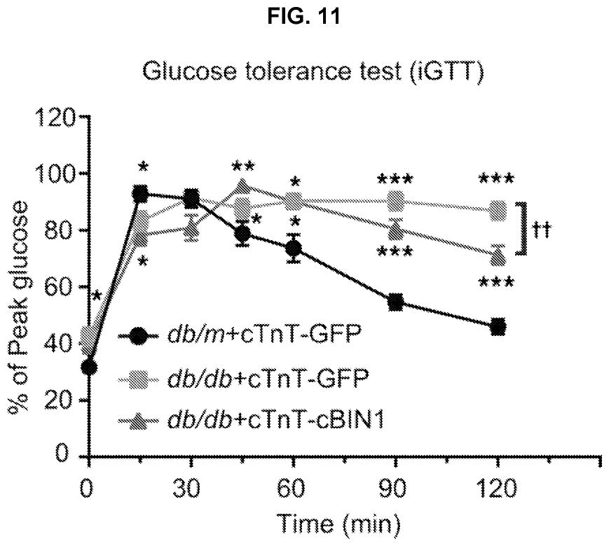

To test for cardiac specificity of the glucose response, gene therapy was then applied with a vector that drives cBIN1 expression by the cardiac-specific promotor chicken cardiac troponin T (cTnT), and the functional analysis was repeated in db/db mice (n=12 per group). As indicated in FIG. 11, AAV9-cTnT-cBIN1 treatment in db/db mice resulted in an increased ability to clear a 1 g/kg glucose load, recapitulating the glycemic control effect induced by low-dose AAV9-CMV-cBIN1 (FIG. 1F). Furthermore, following the same in vivo gene therapy protocol illustrated in FIG. 1A, it was identified that AAV9-cTnT-cBIN1 (1×10¹¹ vg) also provided functional rescue of exercise capacitance and cardiac functional parameters in db/db mice (Table 4).

TABLE 4
Echocardiography parameters and treadmill performance in 17-weeks old
db/m and db/db mice at 8-weeks post AAV9-cTnT-GFP/cBIN1 injection.

| | db/m-GFP | db/db-GFP | db/db-cBIN1 |
|---|---|---|---|
| | (n = 12) | (n = 12) | (n = 12) |
| E (mm/s) | 559.2 ± 36.55 | 397.7 ± 28.52*** | 536.1 ± 25.24^{††} |
| A (mm/s) | 337.5 ± 29.06 | 351.9 ± 24.05 | 355.6 ± 21.06 |
| e′ (mm/s) | 18.11 ± 1.38 | 9.97 ± 0.87*** | 19.48 ± 0.86^{†††} |
| E/A | 1.70 ± 0.07 | 1.14 ± 0.05*** | 1.53 ± 0.06^{††} |
| E/e′ | 32.56 ± 2.74 | 42.88 ± 5.07 | 28.11 ± 1.87^{††} |
| MPI | 0.49 ± 0.02 | 0.79 ± 0.04*** | 0.49 ± 0.01^{†††} |
| IVRT (ms) | 15.20 ± 0.66 | 26.70 ± 1.47*** | 16.15 ± 0.34^{†††} |
| EF (%) | 74.68 ± 2.33 | 74.65 ± 2.80 | 78.32 ± 1.82 |
| LVEDV (μL) | 49.31 ± 3.03 | 35.50 ± 2.42*** | 46.29 ± 1.99^{††} |
| LVESV (μL) | 13.03 ± 1.74 | 9.38 ± 1.55 | 10.16 ± 1.00 |
| HR (bpm) | 403.5 ± 10.60 | 394.6 ± 14.77 | 421.6 ± 11.82 |
| SV (μL) | 36.28 ± 1.73 | 26.12 ± 1.68*** | 36.13 ± 1.56^{†††} |
| CO (mL/min) | 16.50 ± 0.69 | 10.42 ± 0.56*** | 15.45 ± 0.65^{†††} |
| LVAWs (mm) | 1.58 ± 0.06 | 1.56 ± 0.07 | 1.70 ± 0.06 |
| LVAWd (mm) | 0.96 ± 0.04 | 0.95 ± 0.05 | 0.98 ± 0.06 |
| LVPWs (mm) | 1.29 ± 0.08 | 1.27 ± 0.05 | 1.36 ± 0.06 |
| LVPWd (mm) | 0.87 ± 0.05 | 0.83 ± 0.03 | 0.87 ± 0.03 |
| RWT | 0.50 ± 0.03 | 0.51 ± 0.04 | 0.47 ± 0.02 |
| LVM (mm) | 116.7 ± 5.20 | 103.3 ± 4.46 | 128.9 ± 5.30^{†††} |
| BW (g) | 27.51 ± 1.31 | 49.18 ± 0.90*** | 48.11 ± 0.99*** |
| Maximal Distance (m) | 208.8 ± 4.80 | 44.26 ± 4.63*** | 80.98 ± 7.96***^{, †††} |

E, peak velocity blood flow from left ventricular relaxation in early diastole; A, peak velocity flow in late diastole caused by atrial contraction; e′, early diastolic mitral annular tissue velocity; MPI, myocardial performance index; IVRT, isovolumic relaxation time; EF, ejection fraction; LVEDV, left ventricular end-diastolic volume; LVESV, left ventricular end-systolic volume; HR, heart rate; SV, stroke volume; CO, cardiac output; LVAWs, left ventricular end-systolic anterior wall thickness; LVAWd, left ventricular end-diastolic anterior wall thickness; LVPWs, left ventricular end-systolic posterior wall thickness; LVPWd, left ventricular end-diastolic posterior wall thickness; RWT, relative wall thickness; LVM, left ventricular mass; BW, body weight; HW/TL, heart weight/ tibial length; and LW/TL, lung weight/tibial length.
Data are presented as mean ± SEM. One-way ANOVA followed by Bonferroni's test or Kruskal-Wallis test followed by Dunn's test was performed for selected pair comparison.
*, **, ***p < 0.05, 0.01, or 0.001 for comparison vs. db/m-GFP; ^{†}, ^{††}, ^{†††}p < 0.05, 0.01, or 0.001 for comparison vs. db/db-GFP.

Together, these data suggest that cardiomyocyte membrane microdomains, by facilitating GLUT4 translocation to t-tubules, are critical to insulin-stimulated glucose uptake. Beyond cardiac hemodynamic protection, systemic glycemic control also benefited from cBIN1 gene therapy in the heart.

### Example 6

Intravenous AAV9-cBIN1 Transduces Exogenous cBIN1 in Large Animal Hearts

The data of the preceding examples indicate a mechanistic role for cBIN1-organized cardiac microdomains in the hemodynamic and glycemic recovery of diabetic mice. To explore the translational possibility of cBIN1 gene therapy, the cardiac transduction efficiency of AAV9-cBIN1 was tested in a preclinical large-animal minipig model.

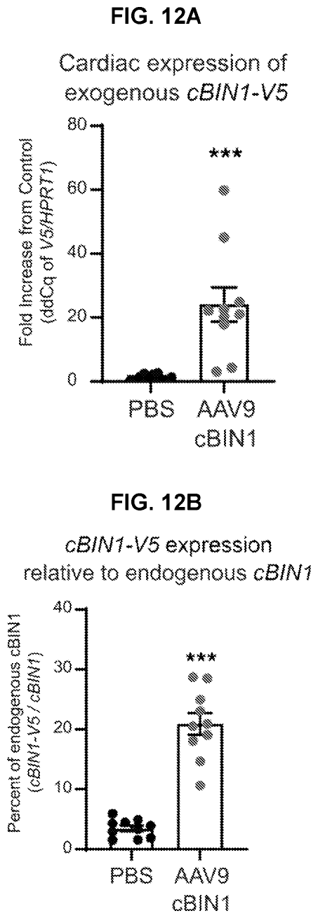

A single i.v. injection of low-dose (i.e., 6×10¹¹ vg/kg) AAV9-cBIN1 was introduced to the minipigs. Six months after injection, animals were terminated and myocardial tissues across left ventricles (apex, anterior wall, posterior wall, base, and septum) were obtained for mRNA extraction and cDNA preparation. Using qPCR with the same V5 Taqman probe used for cBin1-V5 detection in mouse hearts (FIG. 1H), it was confirmed that exogeneous cBin1-V5 expression can be successfully transduced by AAV9-cBIN1 in minipig hearts. Using the ΔΔCt results of V5/HPRT1 compared with control samples, it was found that AAV9 transduced exogenous cBIN1-V5 expressed 24.1-fold±5.4-fold above background, relative to control PBS-treated animal hearts (FIG. 12A), indicating that a single i.v. therapy can last at least 6 months in cardiomyocytes.

Exogenous transduced cBin1 expression versus endogenous cBIN1 expression was then quantified. A TaqMan probe was designed to detect only the exonal junction sequence between porcine BIN1 exons 13 and 17 (cospliced in cBIN1) without recognition of the exogenous mouse cBin1 sequence transduced by AAV9. Using the ΔCt results of V5/cBIN1 (porcine), it was determined that, at 6 months after injection, exogenous cBin1-V5 was expressed in minipig hearts at 20.9%±1.8% of the endogenous cBIN1 transcript levels (FIG. 12B). Together with the observed 20% reduction in endogenous cBIN1 expression in db/db hearts (FIG. 11), these results indicate that the low-dose systemic delivery of AAV9-cBIN1 can effectively transduce exogenous cBIN1 at a level sufficient to replace a 20% reduction of endogenous cBIN1 expression in failing hearts. Further, this transduction-efficient dose of AAV9-cBIN1 (6×10¹¹ vg/kg, i.v.) is close to the lowest therapeutic effective doses tested in phase I/II trials, and is only 1%-3% of the doses used for phase III trials for AAV-mediated gene therapies for other diseases. Cardiac transduction in a large-animal using doses of virus that are sufficiently low could therefore be achieved to limit off-target and potentially toxic side effects.

These studies identified that, in diabetic mice, cBIN1 gene therapy can restore cardiomyocyte t-tubule microdomains, normalizing cardiomyocyte subcellular architecture. This approach can help rescue diastolic dysfunction and clinical features of heart failure with preserved ejection fraction (HFpEF) and can also rescue a deficit in glucose uptake in diabetic cardiomyocytes. Furthermore, AAV9-cBIN1 gene therapy can improve hyperglycemia in type 2 diabetes mellitus (T2DM) mice. In a large-animal minipig model, i.v. introduction of even lower-dose AAV9-cBIN1 provided sufficient cardiomyocyte transduction to compensate for loss of cBIN1 in diabetic heart failure.

cBIN1 microdomains are responsible for proper intracellular trafficking and organization of calcium handling machinery, including SERCA2a, Cav1.2, and RyR₂, and disruption in cBIN1 microdomain contributes to heart failure pathophysiology. cBIN1 microdomains are a critical trafficking hub for both systolic and diastolic calcium-handling proteins, suggesting that the preservation of cBIN1 microdomains could benefit diabetic failing hearts as well. As discussed herein, in the db/db mice with diabetic heart failure without left ventricular dilation, myocardial cBIN1 was only 20% reduced (FIG. 11), which was enough to impair membrane microdomains without affecting t-tubule lumen size. With disease progression of DCM, cBIN1 may further decline and manifest with t-tubule lumen enlargement, as occurs in nondiabetic dilated cardiomyopathy. Nevertheless, just 20% cBIN1 reduction and impairment of microdomains was already enough to cause diastolic dysfunction (FIG. 4A-D). cBIN1 gene therapy improved lusitropy and normalized EDV in db/db hearts, rescuing CO and improving exercise intolerance (FIGS. 1 and 4).

The data presented herein also surprisingly indicate that cBIN1 microdomains at t-tubules could modulate cardiomyocyte glucose uptake by supporting GLUT4 trafficking and surface translocation (FIGS. 2 and 10), and GLUT4 is believed to be an important regulator of cardiac metabolism in diabetes. Furthermore, by improving insulin-stimulated surface translocation of GLUT4 in cardiomyocytes, AAV9-cBIN1 helped rescue hyperglycemia in db/db mice (FIGS. 1 and 11).

The heart is not normally considered a major contributor to modulating blood glucose levels, yet even cardiac-specific expression of cBIN1 was found to reduce hyperglycemia (FIG. 11). Furthermore, data from isolated adult mouse cardiomyocytes indicated that cBIN1 promoted insulin-stimulated glucose uptake by db/db cardiomyocytes, an effect quantitatively similar to that induced by metformin treatment (FIG. 2E). The role of cBIN1 microdomains in regulating insulin-stimulated cardiomyocyte glucose utilization was further supported by impaired intracellular GLUT4 trafficking, 2DG uptake, and 13C6-glucose uptake and oxidation in Bin1 HT cardiomyocytes together with rescue by AAV9-cBIN1 (FIG. 10A-C). In db/db mice with T2DM having already-elevated plasma insulin levels, cBIN1-increased cardiomyocyte insulin sensitivity suggests that heart muscle served as a tissue clearance pathway for systemic glucose control. The altered blood glucose response following insulin administration in aged Bin1 HT mice with a cardiac-specific deletion of Bin1 (FIG. 10) further supports a possible role of insulin-stimulated cardiomyocyte glucose uptake in the regulation of systemic glycemic control. Therefore, cBIN1 gene therapy could provide a promising approach to lower blood glucose in T2DM. Given that skeletal t-tubules express endogenous BIN1, and that insulin-stimulated GLUT4 translocation increases glucose utilization in skeletal muscle, it is possible that skeletal muscle transduction of cBIN1 or the equivalent skeletal isoform of BIN1 may provide additional benefit in hyperglycemic control in T2DM.

In the described minipig study, low-dose cBIN1 both potentially normalized reduced cBIN1 levels in failing hearts (FIGS. 1 and 12B) and maintained expression for at least 6 months (FIG. 12). Much of the toxicity of AAV-based gene therapy is dose dependent and associated with hepatic dysfunction. Low-dose therapy, however, such as that less than 1×10¹² vg/kg, offered significant potential to translate cBIN1 therapy to humans (FIG. 12). In general, introducing a trafficking-related protein can achieve a desired phenotype yet at a low dose. For instance, a low-dose introduction of GJA1-20k can reduce ventricular arrhythmia in mouse models of arrhythmogenic cardiomyopathy. Trafficking-related proteins may be attractive targets for multiple gene therapy solutions to cardiac disease. Additional therapeutic efficacy studies in preclinical large-animal models will evaluate the therapeutic potential of exogenous cBIN1 for patients with T2DM and diabetic cardiomyopathy. The data described herein indicate that cBIN1 microdomains at t-tubules serve as a multifunctional hub modulating cardiomyocyte function and glycemic control.

### Example 7

Dosing Regimen of cBIN1 Gene Therapy for Human Subjects

For systemic glycemic control, cBIN1 gene therapy constructs may be administered to mammalian human subjects using a distinct dosing regimen and delivery route. Based on cBIN1 gene therapy studies in db/db diabetic mice and minipig models, systemic delivery of AAV9-cBIN1 to humans may be employed via a single intravenous (i.v.) bolus injection at a dose calculated based on body weight (vector genome (vg) per kg body weight).

Data from the db/db mice studies indicate that a single i.v. dose of about 2×10¹² vg/kg cBIN1 is effective in reducing blood glucose levels and improving glucose tolerance. Further, data from minipigs with heart failure indicate that a single i.v. dose of about 6×10¹¹ vg/kg cBIN1 is effective in normalizing cardiac function. Thus, for glycemic control, a dosing regimen of AAV9-cBIN1 at a low dose between about 5×10¹¹ vg/kg to about 5×10¹³ vg/kg may be used. This dose may be diluted in 5-10 mL PBS or saline and administered as a single i.v. bolus. For example, for a 70 kg mammalian human subject, a total of about 3.5×10¹³ vg to about 3.5×10¹⁵ vg of AAV9-cBIN1 may be diluted in 10 mL saline and administered as a single i.v. bolus injection. Given the relatively low rates of cardiomyocyte turnover in adult human hearts, the disclosed methods and compositions may provide long-term, durable transgene expression of cBIN1 in large mammalian hearts using these very low doses of i.v. administered cBIN1 gene therapy.

What is claimed:

1. A method of treating, preventing, reducing the likelihood of having, reducing the severity of, and/or slowing the progression of diabetic cardiomyopathy in a subject, the method comprising:
administering to the subject a therapeutically effective amount of a pharmaceutical composition comprising:
a cardiac bridging integrator 1 (cBIN1) gene expression vector comprising a cBIN1 polynucleotide sequence encoding a cBIN1 polypeptide or functional variant or fragment thereof.

2. The method of claim 1, wherein the pharmaceutical composition is administered to the subject by intravenous (i.v.) injection.

3. The method of claim 1, wherein the pharmaceutical composition is administered to the subject by cardiac catheter infusion to myocardium.

4. The method of claim 1, wherein the cBIN1 polynucleotide sequence comprises DNA, RNA, or a combination thereof.

5. The method of claim 1, wherein the cBIN1 polynucleotide sequence has at least 90-99% identity to any one of SEQ ID NO: 1, 2, or 4.

6. The method of claim 1, wherein the cBIN1 polynucleotide sequence is any one of SEQ ID NO: 1, 2, or 4.

7. The method of claim 1, wherein the cBIN1 gene expression vector is selected from a non-viral vector, a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

8. The method of claim 7, wherein the cBIN1 gene expression vector is an AAV vector of a serotype selected from AAV1, AAV2, AAV3, AAV4, AAV5, AAV6, AAV7, AAV8, AAV9, AAV10, AAV11, AAV12, AAVrh74, a hybrid serotype thereof, or a derivative thereof.

9. The method of claim 8, wherein the cBIN1 gene expression vector is an AAV9 vector.

10. The method of claim 8, wherein the cBIN1 gene expression vector is a muscle-tropic AAV vector.

11. The method of claim 7, wherein the non-viral vector comprises a lipid carrier, an exosome, a polymer-based carrier, a chemical-based carrier, a conjugated carrier, or combinations thereof.

12. The method of claim 1, wherein the pharmaceutical composition is administered to the subject using a dosing regimen based on cBIN1 gene expression vector genome (vg) per kg body weight of the subject.

13. The method of claim 12, wherein the pharmaceutical composition is administered to the subject at a dose ranging from about 5×10¹¹ vg/kg to about 5×10¹³ vg/kg cBIN1 gene expression vector.

14. The method of claim 1, wherein the subject has type 1 diabetes or type 2 diabetes.

15. The method of claim 1, wherein the subject has diabetic cardiomyopathy.

16. The method of claim 1, wherein the pharmaceutical composition reduces blood glucose levels in the subject.

17. The method of claim 1, wherein the pharmaceutical composition normalizes or restores the intracellular distribution of calcium handling machinery in myocardium of the subject, wherein the calcium handling machinery comprises one or more of SERCA2a, Cav1.2, or RyR₂.

18. The method of claim 1, wherein the pharmaceutical composition rehabilitates or increases transverse-tubule microfolds or microdomains in myocardium of the subject.

19. The method of claim 1, wherein the pharmaceutical composition selectively expresses the cBIN1 polypeptide or functional variant or fragment thereof in the heart of the subject.

20. The method of claim 19, wherein the pharmaceutical composition increases selective expression of the cBIN1 polypeptide or functional variant or fragment thereof in the heart of the subject by at least about 20% relative to an endogenous cBIN1 expression level in the heart of the subject.

21. The method of claim 1, wherein the pharmaceutical composition expresses the cBIN1 polypeptide or functional variant or fragment thereof in the subject for at least 6 months.

22. The method of claim 1, wherein the subject is a mammal.

23. The method of claim 1, wherein the subject is a human.

24. A pharmaceutical composition comprising a cardiac bridging integrator 1 (cBIN1) gene expression vector comprising a cBIN1 polynucleotide sequence encoding a cBIN1 polypeptide or functional variant or fragment thereof.

25. The pharmaceutical composition of claim 24, wherein the cBIN1 polynucleotide sequence comprises DNA, RNA, or a combination thereof.

26. The pharmaceutical composition of claim 24, wherein the cBIN1 polynucleotide sequence has at least 90-99% identity to any one of SEQ ID NO: 1, 2, or 4.

27. The pharmaceutical composition of claim 24, wherein the cBIN1 polynucleotide sequence is any one of SEQ ID NO: 1, 2, or 4.

28. The pharmaceutical composition of claim 24, wherein the cBIN1 gene expression vector is selected from a non-viral vector, a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

29. The pharmaceutical composition of claim 28, wherein the cBIN1 gene expression vector is an AAV vector of a serotype selected from AAV1, AAV2, AAV3, AAV4, AAV5, AAV6, AAV7, AAV8, AAV9, AAV10, AAV11, AAV12, AAVrh74, a hybrid serotype thereof, or a derivative thereof.

30. The pharmaceutical composition of claim 29, wherein the cBIN1 gene expression vector is an AAV9 vector.

31. The pharmaceutical composition of claim 29, wherein the cBIN1 gene expression vector is a muscle-tropic AAV vector.

32. The pharmaceutical composition of claim 28, wherein the non-viral vector comprises a lipid carrier, an exosome, a polymer-based carrier, a chemical-based carrier, a conjugated carrier, or combinations thereof.

33. Use of a cardiac bridging integrator 1 (cBIN1) gene therapy in a medicament for treating, preventing, reducing the likelihood of having, reducing the severity of, and/or slowing the progression of diabetic cardiomyopathy in a subject.

34. The use of claim 33, wherein the cBIN1 gene therapy comprises a therapeutically effective amount of a pharmaceutical composition comprising:
a cBIN1 gene expression vector comprising a cBIN1 polynucleotide sequence encoding a cBIN1 polypeptide or functional variant or fragment thereof.

35. Use of a plasma cardiac bridging integrator 1 (cBIN1) score (CS) to identify a subject having diabetic cardiomyopathy for cBIN1 gene therapy treatment.

36. The use of claim 35, wherein the cBIN1 gene therapy treatment comprises a therapeutically effective amount of a pharmaceutical composition comprising:
a cBIN1 gene expression vector comprising a cBIN1 polynucleotide sequence encoding a cBIN1 polypeptide or functional variant or fragment thereof.

37. The use of claim 35, wherein the plasma CS is a non-invasive measure of target engagement and therapeutic response of the subject to the cBIN1 gene therapy treatment.

38. The use of claim 35, wherein the plasma CS is the natural log of the ratio of a median plasma cBIN1 concentration in a normal human population to a measured cBIN1 concentration in the subject having diabetic cardiomyopathy.
